(19) United States
(12) Patent Application Publication
(10) Pub. No.: US 2024/0409627 A1
(43) Pub. Date: Dec. 12, 2024

(54) INHIBIN ANTIBODIES AND CANCER TREATMENT

(71) Applicant: THE UAB RESEARCH FOUNDATION, Birmingham, AL (US)

(72) Inventors: Mythreye Karthikeyan, Birmingham, AL (US); Ben Horst, Birmingham, AL (US)

(73) Assignee: THE UAB RESEARCH FOUNDATION, Birmingham, AL (US)

(21) Appl. No.: 18/700,754

(22) PCT Filed: Oct. 18, 2022
(86) PCT No.: PCT/US2022/047036

§ 371 Date: Apr. 12, 2024

Related U.S. Application Data
(60) Provisional application No. 63/256,748, filed on Oct. 18, 2021.

(51) Int. Cl. C07K 16/26; A61K 39/00; A61K 45/06; A61P 35/00

(52) U.S. Cl. CPC C07K 16/26; A61K 45/06; A61P 35/00; A61K 2039/505

(57) ABSTRACT

Disclosed is a method for reducing metastasis and/or tumor growth in a subject with cancer by administering an effective amount of an inhibin antibody to the subject with cancer. In particular, the subject may have ovarian cancer and the administration of inhibin antibody may impair tumor growth.

### PRIORITY CLAIM

This application claims priority to U.S. Provisional Application No. 63/256,738, filed on Oct. 18, 2021, the entire contents and disclosure of which are incorporated herein in their entirety.

### SEQUENCE LISTING

The instant application contains a Sequence Listing which has been submitted electronically in XML file format and is hereby incorporated by reference in its entirety. Said XML copy, created on Oct. 18, 2022, is named 035979-1354626-213WO1.xml and is 23,893 bytes in size.

### STATEMENTS REGARDING FEDERALLY FUNDED RESEARCH

This Invention was made with government support (NIHR01CA219495).

### FIELD OF THE INVENTION

The present invention relates to methods for reducing metastasis, and/or tumor growth and/or altering the vasculature in a subject with cancer, the method comprising administering an effective amount of an inhibin antibody to the subject with cancer. In particular, the subject may have a gynecological cancer and the administration of inhibin antibody may impair tumor growth or/and change the vasculature.

### BACKGROUND

Changes in angiogenesis or blood vessels and the associated vasculature are associated with tumor growth and metastasis in most cancers, including gynecological cancers such as ovarian cancer, with significant impact on tumor progression and ascites development in advanced disease (1, 2). As such, anti-angiogenic therapies have had significant impact in the management of ovarian cancers (3). However, their effectiveness can be frequently limited due in part to toxicities and acquired resistance, leading to challenges with long term use and marginal improvements in overall survival (3). Discovery of new and safer angiogenic targets is thus critical.

TGFβ family members, particularly BMP9 and TGFβ, are the most examined regulators of angiogenesis but have not been effective as targets for angiogenic therapy due to their pleiotropic functions in cancer and normal physiology (4,5). Similar to TGFβ and BMP9, activins have controversial and context dependent roles in angiogenesis. Specifically, activin A has been shown to increase VEGF induced angiogenesis in some instances (6) and in others, has been demonstrated to inhibit angiogenesis (7). Inhibins are a distinct and unique member of the TGFβ family as the only endocrine hormone and a functional heterodimer of an alpha (α) subunit (INHA) and a beta (β) activin subunit (INHBA or INHBB) forming either inhibin A or inhibin B respectively (8). Inhibins are distinct from activins which are comprised of dimers of either beta subunit (8). Inhibin α is synthesized as a pro-peptide with a pro-domain, αN region, and αC region. The pro-domain and αN region can be cleaved to produce the mature Inhibinα subunit comprising the αC region. Physiological Inhibin α production by the sertoli cells of the testes, granulosa cells of the ovary, and the adrenal and pituitary glands (9) is regulated primarily by follicle stimulating hormone (FSH) and luteinizing hormone (LH) (10-11) via a cAMP-PKA (cyclic adenosine monophosphate-protein kinase A) pathway resulting in cAMP response element binding (CREB) to the cAMP response element (CRE) on the INHA promoter (12).

While inhibin levels (inhibin A and B) cycle across the lifespan of healthy females and dramatically decrease at the onset of menopause (13), elevated inhibinα levels are found in ovarian, gastric, hepatocellular, and prostate cancers (14-17). Total inhibin protein levels comprising free inhibinα, inhibin A and inhibin B are also an established diagnostic marker alone and/or in combination with CA125, for ovarian cancers (18) and have been proposed as a potential tumor specific target for therapy (8, 15, 17, 19-20). Inhibin α levels are also predictive of survival in multiple cancer types with gene signatures that correlate with INHA expression, providing a highly accurate prognostic model for predicting patient outcomes (20). However, the mechanism of inhibin expression in cancers have not been delineated.

A need remains for understanding the mechanism of inhibin expression in cancers and for methods for treating cancer and/or tumor growth by administering inhibin antibodies.

### SUMMARY

The terms “invention,” “the invention,” “this invention” and “the present invention” used in this patent are intended to refer broadly to all of the subject matter of this patent and the patent claims below. Statements containing these terms should be understood not to limit the subject matter described herein or to limit the meaning or scope of the patent claims below. Embodiments of the invention covered by this patent are defined by the claims below, not this summary. This summary is a high-level overview of various embodiments of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification of this patent, any or all drawings and each claim.

In some embodiments, the present disclosure is directed to a method of reducing metastasis and/or tumor growth and changing the vasculature in a subject with cancer, the method comprising administering an effective amount of an inhibin antibody to the subject with cancer. The cancer may be selected from the group consisting of breast cancer, gynecological cancers including ovarian cancer, prostate, melanoma, squamous cell carcinoma, bladder cancer, lung cancer, testicular cancer, kidney cancer, colorectal cancer, and head and neck cancer. In some aspects, the cancer is gynecological cancer, such as ovarian cancer. The method may further comprise concurrent administration of an anti-angiogenic. The method may further comprise administration of an anti-angiogenic prior to administration of the inhibin antibody. The method may further comprise administration of an anti-angiogenic after administration of the inhibin antibody. The anti-inhibin may be administered to a subject with tumors resistant to treatment with TRC105 and Becacizumab. The tumor growth may be hypoxia and/or HIF1/2 mediated tumor growth. The inhibin antibody may be anti-inhibin R1 antibody. The inhibin antibody may be PO23 anti-inhibin antibody. The subject may be pre- or post-menopausal. The method may be for reducing metastasis, tumor size or changing the vasculature. The method may be for reducing tumor growth. The method may be for reducing metastasis in a subject with gynecological cancers. The method may be for changing the vasculature. The method may be for reducing tumor growth in a subject with gynecological cancers. The method may further comprise treating the subject with chemotherapy or anti-angiogenic therapy. The subject may be a mammal. In some aspects, the subject is a human. The anti-inhibin may be administered for a period of at least 7 days. The anti-inhibin may be administered in an amount from 0.01 to 200 mg/kg. The method may further comprise concurrent administration of HIF targeted therapy.

### BRIEF DESCRIPTION OF THE DRAWINGS

In the following detailed description, embodiments of the invention are described referring to the following figures (also referred to as FIG. or FIGS. herein):

FIG. 1A shows a qRT-PCR analysis of (i) INHA and (ii) VEGFA mRNA expression normalized to levels in 20% O₂ in HEY and OV90 cells exposed to indicated oxygen concentration for 24 hrs in accordance with embodiments of the disclosure.

FIG. 1B shows a qRT-PCR analysis of (i) INHA and (ii) VEGFA mRNA expression normalized to corresponding levels in normoxia in indicated cells grown under hypoxia (0.2%) or normoxia (17-21%) for 24 h except for OVCAR5 (12 h) in accordance with embodiments of the disclosure.

FIG. 1C shows a total inhibin ELISA (inhibin A/B, inhibinα) of conditioned media collected from OV90 and HEY cells grown in normoxia or after 24 h exposure to hypoxia (0.2% O₂) in accordance with embodiments of the disclosure.

FIG. 1D shows (i) Western blot of HIF-1α levels in HEY and OV90 following exposure to hypoxia (0.2% O₂) for 24 h and after indicated reoxygenation times, (ii) relative qRT-PCR analysis of INHA expression in HEY and OV90 cells following exposure to hypoxia (0.2% O₂) and indicated reoxygenation time normalized to corresponding levels in normoxia in accordance with embodiments of the disclosure.

FIG. 2A shows (i) Western blotting of HIF-1α protein from indicated cells grown in either 2D or under anchorage independence (3D) conditions, vinculin is loading control and (ii) relative qRT-PCR of INHA mRNA expression in OVCA420 and PA1 after 48 h (OVCA420) or 72 h (PA1) of growth under anchorage independence (3D).

FIG. 2B shows total inhibin ELISA of ascites fluid from 25 ovarian cancer patients sorted by stage in accordance with embodiments of the disclosure.

FIG. 2C shows (i) percent hypoxic area in tumors of indicated size range determined by quantitation of pimonidazole staining in tumors in accordance with embodiments of the disclosure.

FIG. 2D shows a correlation analysis between INHA expression and either (i) Buffa or (ii) Winter hypoxia scores from TCGA OVCA (i-ii) or breast (iii) cancer patient data sets from cBioportal measured by RNA-Seq in accordance with embodiments of the disclosure.

FIG. 3A shows (i) Western blot of HIF-1α at indicated time points after treatment with 100 μM CoCl₂ and (ii) a qRT-PCR analysis of INHA and VEGF mRNA in OVCAR5 and PA1 cells after indicated time of treatment with 100 μM of CoCl₂ normalized to untreated in accordance with embodiments of the disclosure.

FIG. 3B shows a qRT-PCR analysis of INHA and ARNT mRNA in HEY shControl or shARNT cell lines after exposure to hypoxia (0.2% O₂) for 24 h normalized to corresponding shControl normoxia levels in accordance with embodiments of the disclosure.

FIG. 3C shows a western blot (above) and relative qRT-PCR analysis of INHA expression (below) from (i) HEY or (ii) OV90 cells transfected with either siScr, siHIF-1α, siHIF-2α, or a combination of siHIF-1/2α and exposed to hypoxia (0.2% O₂) for 24 h in accordance with embodiments of the disclosure.

FIG. 3D shows a qRT-PCR analysis using primers that amplify the proximal HRE region in the INHA promoter after chromatin immunoprecipitation (ChIP) of HIF-1α in OVCAR5 and OV90 cells in accordance with embodiments of the disclosure.

FIG. 3E shows luciferase activity of HEK293 cells transfected with the INHA promoter driven luciferase reporter construct (pGL4.10) and a SV-40 renilla control vector in accordance with embodiments of the disclosure.

FIG. 4A shows (i) hemoglobin content in Matrigel plugs collected 12 days after subcutaneous injection of HEY conditioned media collected from cells exposed to normoxia or hypoxia for 24 h and mixed with either 2 μg of IgG or anti-inhibin R1 antibody. Mean±SEM, n=6 plugs per condition. n.s., not significant; ***p<0.001, One-way ANOVA followed by Tukey's multiple comparison test. (ii) Representative images of Matrigel plugs from (i) Scale bar: 2 mm.

FIG. 4B shows a quantitation of HMEC-1 migration through fibronectin coated 8 μm trans-well filter (i-ii) towards conditioned media from OV90 or HEY cells exposed to hypoxia (0.2% O₂) with either 2 μg of R1 or PO23 anti-inhibin antibody or IgG as a control, or towards (iii) serum free media containing 1 nM inhibin A or 1 nM VEGFA. Nuclei from three representative fields per filter were counted in accordance with embodiments of the disclosure.

FIGS. 4C and 4D shows a quantitation of endothelial cell permeability by measuring FITC-dextran changes across a HMEC-1 monolayer in accordance with embodiments of the disclosure.

FIG. 4E shows a HEY trans-endothelial migration (TEM) across HMEC-1 monolayer either treated with inhibin A for 4 h or untreated in accordance with embodiments of the disclosure.

FIG. 5A shows (i) representative immunofluorescence images of F-actin (red) or VE-Cadherin (green), (ii) quantitation of actin stress fibers from in accordance with embodiments of the disclosure.

FIG. 5B shows (i) Western blot analysis of pMLC-2 from HMEC-1 cells and (ii) quantitation of pMLC-2 changes in (i) in accordance with embodiments of the disclosure.

FIG. 5C shows (i) a schematic of VE-cadherin internalization (ii) representative immunofluorescent images, (iii) internalized VE-Cadherin at 37° C. detected with a FITC-secondary antibody in accordance with embodiments of the disclosure.

FIG. 6A shows a quantitation of endothelial cell permeability by measuring FITC-dextran changes across a HMEC-1 monolayer treated with 1 nM inhibin A in accordance with embodiments of the disclosure.

FIG. 6B shows internalization of VE-cadherin measured by cell surface biotinylation in accordance with embodiments of the disclosure.

FIGS. 6C-D show Patch/FRAP studies on the effect of inhibin A on endoglin-ALK1 (c) and endoglin-ALK4 (d) complex formation in accordance with embodiments of the disclosure.

FIG. 7A shows growth curves of subcutaneously implanted HEY shControl or shINHA tumors exposed to either normoxia or hypoxia (0.2% O₂) 24 h prior to injection in accordance with embodiments of the disclosure.

FIG. 7B shows fold change of proteins most altered in shControl and shINHA tumors in accordance with embodiments of the disclosure.

FIG. 7C shows (i) average tumor volume and quantitation of extravasated rhodamine-dextran (in accordance with embodiments of the disclosure.

FIG. 7D shows quantitation of average (i) vessel number and (ii) size in a 10× field using ImageJ (Methods), and (iii) representative images of CD-31 (red) staining in HEY shControl and shINHA subcutaneous tumors in accordance with embodiments of the disclosure.

### DETAILED DESCRIPTION

The subject matter of embodiments of the present invention is described here with specificity to meet statutory requirements, but this description is not necessarily intended to limit the scope of the claims. The claimed subject matter may be embodied in other ways, may include different elements or steps, and may be used in conjunction with other existing or future technologies. This description should not be interpreted as implying any particular order or arrangement among or between various steps or elements except when the order of individual steps or arrangement of elements is explicitly described.

Described herein is a method of reducing metastasis and/or tumor growth in a subject with cancer, by administering an effective amount of an inhibin antibody to the subject with cancer. Without being bound by theory, it is believed that administering an inhibin antibody affects hypoxia, thereby leading to a reduction in metastasis and/or tumor growth.

As used throughout, cancer refers to any cellular disorder in which the cells proliferate more rapidly than normal tissue growth and have altered blood vessels. A proliferative disorder includes, but is not limited to, neoplasms, which are also referred to as tumors. A neoplasm can include, but is not limited to, pancreatic cancer, breast cancer, brain cancer (e.g., glioblastoma), lung cancer, a central nervous system cancer, prostate cancer, colorectal cancer, head and neck cancer, ovarian and related gynecological cancer, thyroid cancer, renal cancer, bladder cancer, adrenal cancer and liver cancer A neoplasm can be a solid neoplasm (e.g., sarcoma or carcinoma) or a cancerous growth affecting the hematopoietic system. In some examples, the cancer is a triple negative (estrogen receptors negative (ER−), progesterone receptors negative (PR−) and HER2 negative (HER2−)) breast cancer. Examples of hematopoietic malignancies include, but are not limited to, myelomas, leukemias, lymphomas (Hodgkin's and non-Hodgkin's forms), T-cell malignancies, B-cell malignancies, and lymphosarcomas. Here, while gynecological cancers are the primary focus, the cancer to be treated is not limited thereto. In some aspects, the cancer is selected from the group consisting of breast cancer, ovarian cancer, melanoma, squamous cell carcinoma, bladder cancer, lung cancer, testicular cancer, kidney cancer, colorectal cancer, and head and neck cancer. In some aspects, the cancer is a gynecological cancer, such as ovarian cancer.

### Treatment

In the methods provided herein, a reduction in metastasis refers to the reduction is the size of tumors, slowing of the spread of cancer cells into the peritoneal cavity, organs including liver, omentum, peritoneum, intestinal lining and/or into lymph nodes and via circulation to lungs and bones. Particularly for ovarian cancer, the initial steps of metastasis are regulated by a controlled interaction of adhesion receptors and proteases, and metastasis is characterized by tumor nodules on mesothelium covered surfaces, causing ascites, bowel obstruction, and tumor cachexia.

The term “treatment”, as used herein, refers to any type of therapy, which aims at terminating, preventing, ameliorating or reducing the susceptibility to a clinical condition as described herein. In a preferred embodiment, the term treatment relates to prophylactic treatment (i.e., a therapy to reduce the susceptibility to a clinical condition), of a disorder or a condition as defined herein. Thus, “treatment,” “treating,” and their equivalent terms refer to obtaining a desired pharmacologic or physiologic effect, covering any treatment of a pathological condition or disorder in a mammal, including a human. The effect may be prophylactic in terms of completely or partially preventing a disorder or symptom thereof and/or may be therapeutic in terms of a partial or complete cure for a disorder and/or adverse effect attributable to the disorder. That is, “treatment” includes (1) preventing the disorder from occurring or recurring in a subject, (2) inhibiting the disorder, such as arresting its development, (3) stopping or terminating the disorder or at least symptoms associated therewith, so that the host no longer suffers from the disorder or its symptoms, such as causing regression of the disorder or its symptoms, for example, by restoring or repairing a lost, missing or defective function, or stimulating an inefficient process, or (4) relieving, alleviating, or ameliorating the disorder, or symptoms associated therewith, where ameliorating is used in a broad sense to refer to at least a reduction in the magnitude of a parameter, such as inflammation, pain, or immune deficiency to a reduction in growth and symptoms an increase in the responsiveness of the cancer to treatment.

Any of the methods provided herein can further comprise administering an anti-cancer compound, e.g., a chemotherapeutic agent, prior to, concurrently or after administration of the inhibin antibody to the subject. Examples of anti-cancer compounds include, but are not limited to avastin, adriamycin, dactinomycin, bleomycin, vinblastine, acivicin, aclarubicin, acodazole hydrochloride, acronine, adozelesin, aldesleukin, altretamine, ambomycin, ametantrone acetate, aminoglutethimide, amsacrine, anastrozole, anthramycin, asparaginase, asperlin, azacitidine, azetepa, azotomycin, batimastat, benzodepa, bicalutamide, bisantrene hydrochloride, bisnafide dimesylate, bizelesin, bleomycin sulfate, brequinar sodium, bropirimine, busulfan, cactinomycin, calusterone, caracemide, carbetimer, carboplatin, carmustine, carubicin hydrochloride, carzelesin, cedefingol, chlorambucil, cirolemycin, cisplatin, cladribine, crisnatol mesylate, cyclophosphamide, cytarabine, dacarbazine, daunorubicin hydrochloride, decitabine, dexormaplatin, dezaguanine, dezaguanine mesylate, diaziquone, doxorubicin, doxorubicin hydrochloride, droloxifene, droloxifene citrate, dromostanolone propionate, duazomycin, edatrexate, eflornithine hydrochloride, elsamitrucin, enloplatin, enpromate, epipropidine, epirubicin hydrochloride, erbulozole, esorubicin hydrochloride, estramustine, estramustine phosphate sodium, etanidazole, etoposide, etoposide phosphate, etoprine, fadrozole hydrochloride, fazarabine, fenretinide, floxuridine, fludarabine phosphate, fluorouracil, flurocitabine, fosquidone, fostriecin sodium, gemcitabine, gemcitabine hydrochloride, hydroxyurea, idarubicin hydrochloride, ifosfamide, ilmofosine, interleukin II (including recombinant interleukin II, or rIL2), interferon alfa-2a, interferon alfa-2b, interferon alfa-n1, interferon alfa-n3, interferon beta-1a, interferon gamma-1 b, iproplatin, irinotecan hydrochloride, lanreotide acetate, letrozole, leuprolide acetate, liarozole hydrochloride, lometrexol sodium, lomustine, losoxantrone hydrochloride, masoprocol, maytansine, mechlorethamine hydrochloride, megestrol acetate, melengestrol acetate, melphalan, menogaril, mercaptopurine, methotrexate, methotrexate sodium, metoprine, meturedepa, mitindomide, mitocarcin, mitocromin, mitogillin, mitomalcin, mitomycin, mitosper, mitotane, mitoxantrone hydrochloride, mycophenolic acid, nocodazole, nogalamycin, ormaplatin, oxisuran, paclitaxel, pegaspargase, peliomycin, pentamustine, peplomycin sulfate, perfosfamide, pipobroman, piposulfan, piroxantrone hydrochloride, plicamycin, plomestane, porfimer sodium, porfiromycin, prednimustine, procarbazine hydrochloride, puromycin, puromycin hydrochloride, pyrazofurin, riboprine, rogletimide, safingol, safingol hydrochloride, semustine, simtrazene, sparfosate sodium, sparsomycin, spirogermanium hydrochloride, spiromustine, spiroplatin, streptonigrin, streptozocin, sulofenur, talisomycin, tecogalan sodium, tegafur, teloxantrone hydrochloride, temoporfin, teniposide, teroxirone, testolactone, thiamiprine, thioguanine, thiotepa, tiazofurin, tirapazamine, toremifene citrate, trestolone acetate, triciribine phosphate, trimetrexate, trimetrexate glucuronate, triptorelin, tubulozole hydrochloride, uracil mustard, uredepa, vapreotide, verteporfin, vinblastine sulfate, vincristine sulfate, vindesine, vindesine sulfate, vinepidine sulfate, vinglycinate sulfate, vinleurosine sulfate, vinorelbine tartrate, vinrosidine sulfate, vinzolidine sulfate, vorozole, zeniplatin, zinostatin, zorubicin hydrochloride.

Any of the methods provided herein can optionally further include administering radiation therapy to the subject. Any of the methods provided herein can optionally further include surgery. Any of the methods provided herein can optionally include administration of an anti-angiogenic, such as concurrent administration. Any of the methods provided herein can optionally include administration of an HIF targeted therapy, such as concurrent administration.

As used throughout, by subject is meant an individual. Preferably, the subject is a mammal such as a primate, and, more preferably, a human. Non-human primates are subjects as well. The term subject includes domesticated animals, such as cats, dogs, etc., livestock (for example, cattle, horses, pigs, sheep, goats, etc.) and laboratory animals (for example, ferret, chinchilla, mouse, rabbit, rat, gerbil, guinea pig, etc.). Thus, veterinary uses and medical formulations are contemplated herein.

Throughout, treat, treating, and treatment refer to a method of reducing or delaying one or more effects or symptoms of cancer or metastasis. In some examples, the cancer is ovarian cancer. Treatment can also refer to a method of reducing the underlying pathology rather than just the symptoms. The effect of the administration to the subject can have the effect of, but is not limited to, reducing one or more symptoms (e.g., reduced pain, reduced size of the tumor, etc.) of the cancer, an increase in survival time, a decrease or delay in metastasis, enhancing T cell function (e.g., proliferation, cytokine production, tumor cell killing), a reduction in the severity of the cancer (e.g., reduced rate of growth of a tumor or rate of metastasis), increasing latency between symptomatic episodes, decreasing the number or frequency of relapse episodes, the complete ablation of the cancer or a delay in the onset or worsening of one or more symptoms. For example, a disclosed method is considered to be a treatment if there is about a 10% reduction in one or more symptoms of the disease in a subject when compared to the subject prior to treatment or when compared to a control subject or control value. Thus, the reduction can be about a 10, 20, 30, 40, 50, 60, 70, 80, 90, 100%, or any amount of reduction in between.

As used throughout by prevent, preventing, or prevention is meant a method of precluding, delaying, averting, obviating, forestalling, stopping, or hindering the onset, incidence, severity, or recurrence of cancer, such as ovarian cancer. For example, the disclosed method is considered to be a prevention if there is a reduction or delay in onset, incidence, severity, or recurrence of cancer, such as gynecological cancer or one or more symptoms of cancer, such as ovarian cancer (e.g., relapse, disease progression, increase in tumor size, metastasis) in a subject treated with inhibin antibody and an anti-cancer compound as compared to control subjects treated with the same anti-cancer compound that did not receive inhibin antibody. The disclosed method is also considered to be a prevention if there is a reduction or delay in onset, incidence, severity, or recurrence of cancer, such as ovarian cancer or one or more symptoms of cancer, such as ovarian cancer in a subject after receiving inhibin antibody as compared to the subject's progression prior to receiving treatment. Thus, the reduction or delay in onset, incidence, severity, or recurrence of a cancer, such as ovarian cancer, can be about a 10, 20, 30, 40, 50, 60, 70, 80, 90, 100%, or any amount of reduction in between.

A biological sample can be any sample obtained from an organism. Examples of biological samples include body fluids and tissue specimens. For example, the sample can be a tissue biopsy, for example, a tumor biopsy. The source of the sample may also be physiological media such as blood, serum, plasma, cerebral spinal fluid, breast milk, pus, tissue scrapings, washings, urine, feces, tissue, such as lymph nodes, spleen, ascites fluid, peritoneal washings or the like. The term tissue refers to any tissue of the body, including blood, connective tissue, epithelium, contractile tissue, neural tissue, and the like.

In the methods provided herein, methods standard in the art for quantitating nucleic acids may be used and are described in detail further herein. These include, but are not limited to, in situ hybridization, quantitative PCR, RT-PCR, Taqman assay, Northern blotting, ELISPOT, dot blotting, ELISA etc., as well as any other method now known or later developed for quantitating the amount of a nucleic acid in a cell or released from a cell. The amount of inhibin antibody can be determined by methods standard in the art for quantitating proteins such as densitometry, absorbance assays, fluorometric assays, Western blotting, ELISA, radioimmunoassay, ELISPOT, immunoprecipitation, immunofluorescence (e.g., FACS), immunohistochemistry, etc., as well as any other method now known or later developed for quantitating a specific protein in or produced by cells in a sample.

The term effective amount, as used throughout, is defined as any amount of an agent (for example, inhibin antibody, a chemotherapeutic agent, etc.) necessary to produce a desired physiologic response. Exemplary dosage amounts for a mammal include doses from about 0.5 to about 200 mg/kg of body weight of active compound per day, which may be administered in a single dose or in the form of individual divided doses, such as from 1 to 4 times per day can be used. Alternatively, the dosage amount can be from about 0.5 to about 150 mg/kg of body weight of active compound per day, about 0.5 to 100 mg/kg of body weight of active compound per day, about 0.5 to about 75 mg/kg of body weight of active compound per day, about 0.5 to about 50 mg/kg of body weight of active compound per day, about 0.5 to about 25 mg/kg of body weight of active compound per day, about 0.5 to about 15 mg/kg of body weight of active compound per day, about 0.5 to about 10 mg/kg of body weight of active compound per day, about 0.5 to about 5 mg/kg of body weight of active compound per day, about 1 to about 20 mg/kg of body weight of active compound per day, about 1 to about 10 mg/kg of body weight of active compound per day, about 1 to about 5 mg/kg of body weight of active compound per day, about 20 mg/kg of body weight of active compound per day, about 10 mg/kg of body weight of active compound per day, or about 5 mg/kg of body weight of active compound per day. One of skill in the art would adjust the dosage as described below based on specific characteristics of the agent and the subject receiving it.

Effective amounts and schedules for administering the agent can be determined empirically and making such determinations is within the skill in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, unwanted cell death, and the like. Generally, the dosage will vary with the type of inhibitor, the species, age, body weight, general health, sex and diet of the subject, the mode and time of administration, rate of excretion, drug combination, and severity of the particular condition and can be determined by one of skill in the art. The dosage can be adjusted by the individual physician in the event of any contraindications. Dosages can vary, and can be administered in one or more dose administrations daily, for one or several days. Guidance can be found in the literature for appropriate dosages for given classes of pharmaceutical products.

In some aspects, the inhibin antibody may be administered in an amount ranging from 0.01 mg/kg to 50 mg/kg, e.g., 0.01 mg/kg, 0.05 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg·kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.7 mg/kg, 4.8 mg/kg, 4.9 mg/kg, 5.0 mg/kg, 6.0 mg/kg, 10 mg/kg, 15 mg/kg, 20 mg/kg, 25 mg/kg, 30 mg/kg, 35 mg/kg, 40 mg/kg, 45 mg/kg, 50 mg/kg, or and combination of values and ranges disclosed herein.

In some aspects, the inhibin antibody may be administered daily for a certain period of time, as such a period of time before or alongside an anti-cancer compound is to be administered. For example, the inhibin antibody may be administered for a period of at least 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or even longer, such as for up to 60 days, 90 days, 120 days, 180 days, or longer. The administration may be once a day, twice a day, or more frequently. There may also be a break between administration, such as a break of last least 12 hours, at least 24 hours, at least 36 hours, at least 48 hours, at least 72 hours, at least 96 hours, at least 120 hours, at least 144 hours, at least one week, at least one week, at least one month, at least 3 months, or at least 6 months. These values may also be used as upper limits for the time between administration.

Any of the agents described herein can be provided in a pharmaceutical composition. These include, for example, a pharmaceutical composition comprising a therapeutically effective amount of one or more agents and a pharmaceutically acceptable carrier.

Depending on the intended mode of administration, the pharmaceutical composition can be in the form of solid, semi-solid or liquid dosage forms, such as, for example, tablets, suppositories, pills, capsules, powders, liquids, or suspensions, preferably in unit dosage form suitable for single administration of a precise dosage. The compositions will include a therapeutically effective amount of the agent described herein or derivatives thereof in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected agent without causing unacceptable biological effects or interacting in a deleterious manner with the other components of the pharmaceutical composition in which it is contained.

As used herein, the term carrier encompasses any excipient, diluent, filler, salt, buffer, stabilizer, solubilizer, lipid, stabilizer, or other material known in the art for use in pharmaceutical formulations. The choice of a carrier for use in a composition will depend upon the intended route of administration for the composition. The preparation of pharmaceutically acceptable carriers and formulations containing these materials is described in, e.g., Remington: The Science and Practice of Pharmacy, 22nd edition, Loyd V. Allen et al, editors, Pharmaceutical Press (2012).

Examples of physiologically acceptable carriers include buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN® (ICI, Inc.; Bridgewater, New Jersey), polyethylene glycol (PEG), and PLURONICS™ (BASF; Florham Park, NJ).

Compositions containing one or more of the agent(s) described herein suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants such as preserving, wetting, emulsifying, and dispensing agents. Prevention of the action of microorganisms can be promoted by various antibacterial and antifungal agents, for example, parabens, chlorobutanol, phenol, sorbic acid, and the like. Isotonic agents, for example, sugars, sodium chloride, and the like may also be included. Prolonged absorption of the injectable pharmaceutical form can be brought about by the use of agents delaying absorption, for example, aluminum monostearate and gelatin.

Solid dosage forms for oral administration of the compounds described herein or derivatives thereof include capsules, tablets, pills, powders, and granules. In such solid dosage forms, the compounds described herein or derivatives thereof are admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others known in the art. They may contain opacifying agents and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration of the compounds described herein or derivatives thereof include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, such as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like.

Besides such inert diluents, the composition can also include additional agents, such as wetting, emulsifying, suspending, sweetening, flavoring, or perfuming agents.

The compositions are administered in any of a number of ways depending on whether local or systemic treatment is desired and on the area to be treated. Any of the compositions described herein can be delivered by any of a variety of routes including by injection (e.g., subcutaneous, intramuscular, intravenous, intra-arterial, intraperitoneal), by continuous intravenous infusion, cutaneously, dermally, transdermally, orally (e.g., tablet, pill, liquid medicine, edible film strip), by implanted osmotic pumps, by suppository, or by aerosol spray. Routes of administration include, but are not limited to, topical, intradermal, intrathecal, intralesional, intratumoral, intrabladder, intravaginal, intra-ocular, intrarectal, intrapulmonary, intracranial, intraventricular, intraspinal, dermal, subdermal, intra-articular, placement within cavities of the body, nasal inhalation, pulmonary inhalation, impression into skin, and electroporation.

In an example in which a nucleic acid is employed the nucleic acid can be delivered intracellularly (for example by expression from a nucleic acid vector or by receptor-mediated mechanisms), or by an appropriate nucleic acid expression vector which is administered so that it becomes intracellular, for example by use of a retroviral vector (see U.S. Pat. No. 4,980,286), or by direct injection, or by use of microparticle bombardment (such as a gene gun; Biolistic, Dupont), or coating with lipids or cell-surface receptors or transfecting agents, or by administering it in linkage to a homeobox-like peptide which is known to enter the nucleus (for example Joliot et al., Proc. Natl. Acad. Sci. USA 1991, 88:1864-8). Nucleic acid carriers also include, polyethylene glycol (PEG), PEG-liposomes, branched carriers composed of histidine and lysine (HK polymers), chitosan-thiamine pyrophosphate carriers, surfactants, nanochitosan carriers, and D5W solution. The present disclosure includes all forms of nucleic acid delivery, including naked DNA, plasmid and viral delivery, integrated into the genome or not. Physical transduction techniques can also be used, such as liposome delivery and receptor-mediated and other endocytosis mechanisms (see, for example, Schwartzenberger et al., Blood 87:472-478, 1996) to name a few examples. This invention can be used in conjunction with any of these or other commonly used gene transfer methods.

Effective doses for any of the administration methods described herein can be extrapolated from dose-response curves derived from in vitro or animal model test systems.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

### Hypoxia

Hypoxia, a driver of tumor growth and metastasis, regulates angiogenic pathways that are targets for vessel normalization and ovarian cancer management. However, toxicities and resistance to anti-angiogenics can limit their use making identification of new targets vital. Inhibin, a heteromeric TGFβ ligand, is a contextual regulator of tumor progression acting as an early tumor suppressor, yet also an established biomarker for ovarian cancers. Without being bound by theory, it is believed that that hypoxia increases inhibin levels in ovarian cancer cell lines, xenograft tumors, and patients. Inhibin is regulated primarily through HIF-1, shifting the balance under hypoxia from activins to inhibins. Hypoxia regulated inhibin promotes tumor growth, endothelial cell invasion and permeability. Targeting inhibin in vivo through knockdown and anti-inhibin strategies robustly reduces permeability in vivo and alters the balance of pro and anti-angiogenic mechanisms resulting in vascular normalization. Mechanistically, inhibin regulates permeability by increasing VE-cadherin internalization via ACVRL1 and CD105, a receptor complex that is shown to be stabilized directly by inhibin. Again, without being bound by theory, it is believed that there are direct roles for inhibins in vascular normalization via TGF-β receptors providing new insights into the therapeutic significance of inhibins as a strategy to normalize the tumor vasculature in gynecological cancers such as ovarian cancer.

Hypoxia is a key mediator of angiogenic responses, regulating pro and anti-angiogenic genes impacting tumor growth, metastasis, and immune evasion (21) and is driven by the hypoxia inducible factor (HIF) family of transcription factors. Hypoxia induced changes, specifically in tumors, are characterized by inefficient oxygen delivery, leading to leaky vessels, and altered permeability, build-up of fluid and ascites in ovarian cancer, and metastasis by facilitating intra/extravasation of tumor cells (21-22). It is known that mice bearing tumor cells with INHA knockdown showed decreased ascites accumulation in (19), indicating a potential role for inhibin in regulating metastasis and vascular functions, a key contributing factor to ascites accumulation. Moreover, inhibin secreted by tumor cells induces angiogenesis via SMAD1/5 signaling in endothelial cells in a paracrine manner dependent on the type III TGFβ receptor endoglin/CD105 and the type I TGFβ receptor ALK1 (19).

To precisely delineate inhibin's significance in cancer and mechanism of action, the inventors have determined the impact of hypoxia, a key mediator of the angiogenic and metastatic response in cancer (21), and the contribution of inhibin to the hypoxia adaptive response. The inventors found that hypoxia in ovarian xenograft tumors, cancer cells, and patient samples leads to an increase in inhibin synthesis in a hypoxia inducible factor (HIF) dependent manner. Thus, hypoxia induced tumor growth and vascular permeability in vivo is driven by inhibin. Moreover, intervention using an antibody based therapeutic strategy to inhibin can suppress hypoxia driven tumor biology. Mechanistically, inhibin promotes vascular permeability via endoglin and ALK1. Notably, using sensitive biophysical methods, the nature and stability of the endoglin and ALK1 interaction at the cell surface in response to inhibin is defined. Inhibins are thus part of the hypoxia adaptive response, and, even further, anti-inhibins may be used as an alternative or companion to current anti-angiogenic therapies that may otherwise not be well tolerated.

Hypoxia significantly impacts several aspects of tumor progression by regulating pathways that can be targeted for cancer management, particularly angiogenic mechanisms. The inventors have identified inhibins, that are biomarkers for ovarian and other cancers and a member of the TGFβ superfamily, to be targets of the hypoxic response. The inventors significantly extended their previous findings (19) to demonstrate that hypoxia induced tumor growth, angiogenesis and vascular leakiness is accompanied with, and dependent on inhibin levels in cells and tumors, and relevant to the ovarian cancer patient population. In keeping with this, hypoxia induced tumor growth can be suppressed by treatment with a selective inhibin antibody that leads to a shift in the angiogenic balance in tumors. The inventors also provide mechanistic evidence for the involvement of ALK1 and CD105/endoglin in inhibin's effects on permeability via increased VE-cadherin internalization. Due to the lack of systemic inhibin expression in post-menopausal women, establishing the therapeutic significance of targeting inhibin in this patient population may be particularly beneficial to evade systemic side effects seen with targeting other hypoxia associated angiogenic pathways.

Significant information exists on the cycling levels of inhibins in premenopausal women, the decline of inhibin during peri-menopause, and as a marker whose decline defines the onset of menopause leading to complete absence of inhibin in normal post-menopausal women (13). Contrastingly, several studies have reported elevated levels of Inhibin in a subset of cancers (14-15, 17, 23). The studies reported herein shed light on the potential mechanisms leading to elevated inhibin. Without being bound by theory, it is believed that total inhibin is elevated in the ascites fluid of patients with ovarian cancer, a hypoxic environment that aids in dissemination of shed ovarian cancer spheroids (22, 30). Serum inhibin and CA125 levels are both markers for ovarian cancer (17-18) and were also positively correlated with each other in these patient ascites. Menopause status was unknown in these patients however the median age of the cohort was 62 and only two patients were below 50 years of age. INHA expression and hypoxia are also correlated through a hypoxia gene score. Supporting the hypothesis that inhibin is regulated by hypoxia, the inventors also found that exposure of ovarian cancer cells to hypoxia increased INHA expression and inhibin secretion most significantly at 0.2% oxygen after 24 h. While a trend towards increase in INHA was seen at higher oxygen tensions (2.5% and 1%), these did not reach significance and could be due to additional time or additional factors needed at higher oxygen levels. Surprisingly and unexpectedly, the inventors did not note consistent and statistically significant increases in the activin subunits, INHBA or INHBB which only appeared to be moderately elevated indicating that increased inhibin secretion levels were driven by inhibinα. Previous reports indicate activin, specifically INHBA, increases in response to hypoxia in endothelial cells (57). However, here the increase in INHA levels in endothelial cells in response to hypoxia was only moderate as compared to in tumor cells suggesting a potential competition in the tumor microenvironment between activin and inhibin. The inhibin ELISA used here does not detect dimeric or free activin, as it is specific to inhibinα and detects all forms of inhibin, namely inhibin A/B and free alpha subunit. Hence, a hypoxia dependent increase in the alpha subunit (INHA), in the absence of a change in activin mRNA levels, could potentially shift the dimerization of the beta subunits (INHBA/INHBB) from activin homodimers to inhibin heterodimers.

Evidence of HIF-1 dependency was observed when hypoxia exposed cells were re-exposed to oxygen (reoxygenation). HIF-1 levels returned to near baseline levels after 1 h which corresponded with INHA expression decreasing to levels not significantly different than baseline normoxic levels. The inventors did observe elevated, although non-significant, INHA in OV90 cells after 1 h of reoxygenation which could be attributed to mRNA turnover mechanisms. Mechanistically, through knockdown studies and ChIP studies, INHA expression is likely regulated through the HIF-1 transcription factor binding directly to the INHA promoter. The findings on a hypoxia response in a pathological condition as seen here, is consistent with a previous report demonstrating that FSH can drive INHA expression in granulosa cells dependent on HIF-1 in what appeared to be in an indirect manner (58). Intriguingly, evidence for INHA regulation by hypoxia, specifically dependent on HIF isoforms, has been demonstrated in cytotrophoblasts (59). As detailed in the Figures and Examples below, there is detailed evidence of regulation by HIF-1, with HIF-1 interacting at INHA's promoter under hypoxia. cAMP and PKA can be activated in response to hypoxia as well. Although the PKA inhibitor, H89, was not able to reduce hypoxia induced INHA expression, indicating that cAMP may not be involved in the hypoxia transcriptional regulation of INHA, this does not preclude a role for cAMP-PKA in the regulation of INHA as it is well-established that the cAMP-PKA signaling axis enhances tumorigenesis in ovarian cancer (60). As the effect of forskolin was additive on INHA expression, cAMP and PKA could represent an alternative or additive mechanism of regulation of INHA in ovarian cancer.

In prostate and adrenocortical cancers reports of both increased and decreased inhibinα levels have been reported (15, 61, 62). In adrenocortical tumors with lower INHA levels, methylation of the INHA promoter was reported to occur at the CpG island within the proximal HRE site identified, suggesting potential roles for epigenetic regulation of INHA as well (62). HIF transcription factors have reduced binding to methylated hypoxia response elements (63). To this end, it is possible that not all cell lines will increase inhibin expression in response to hypoxia. If this is the case, methylation of INHA's promoter may play a role making further understanding of the regulation of INHA expression, particularly in patients necessary in the future.

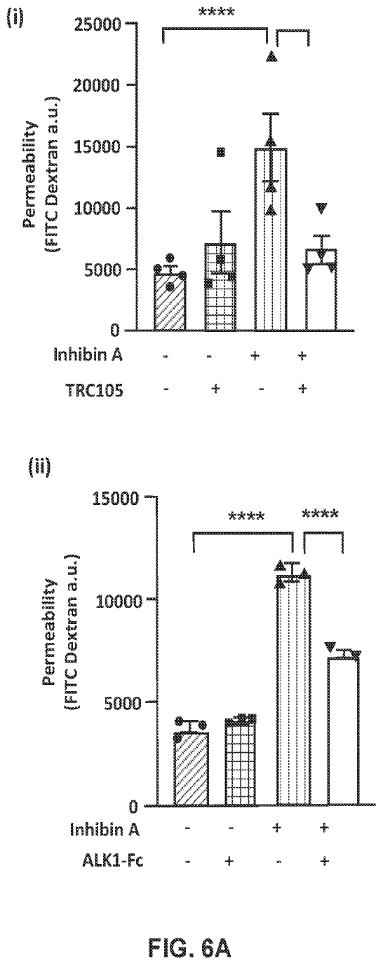

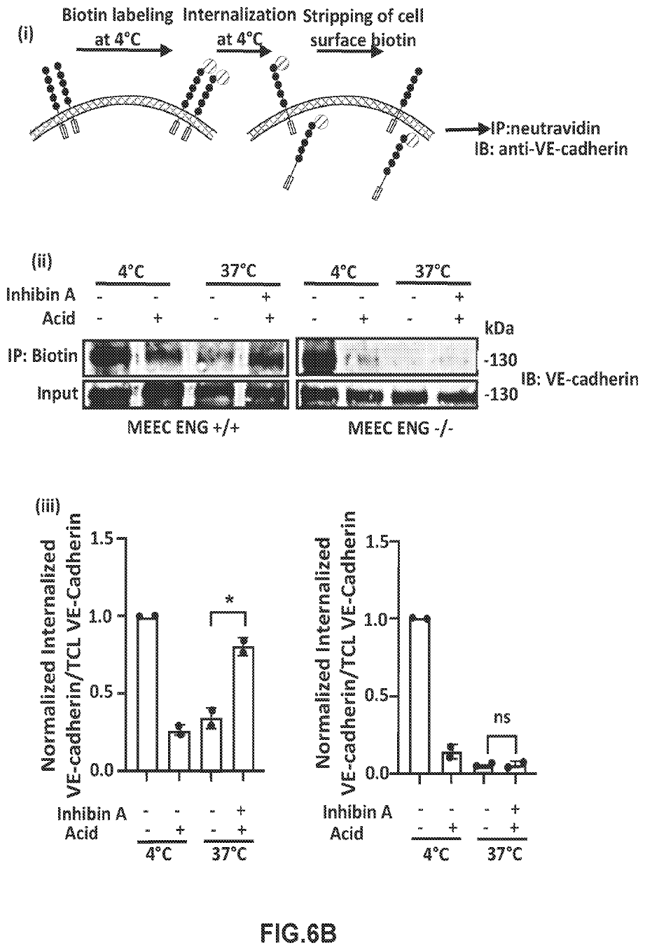

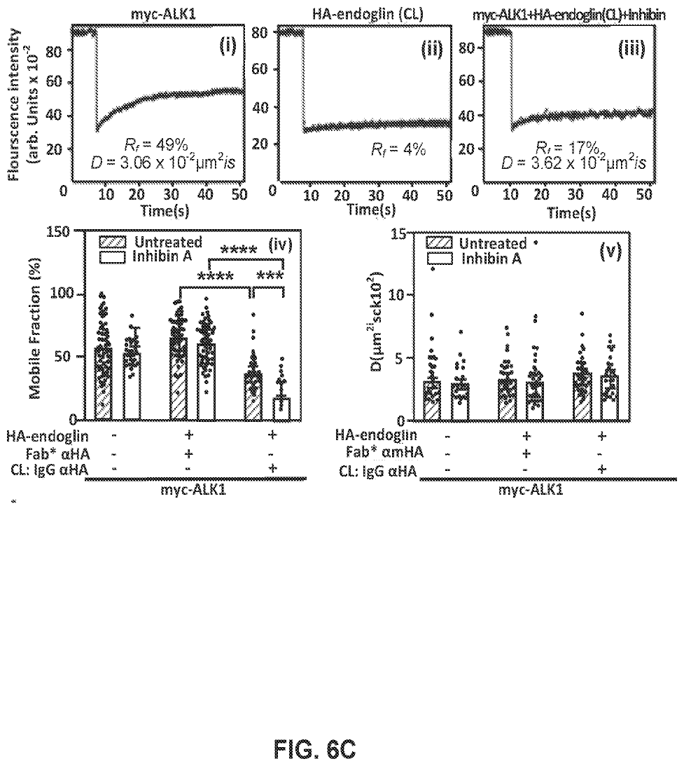

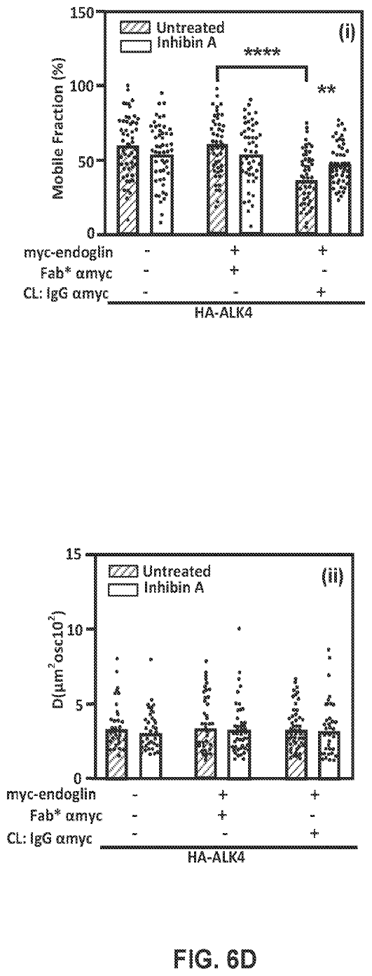

Inhibin's effects have been broadly demonstrated on angiogenesis (19). Here, the outcomes of inhibin's effects on angiogenesis are more precisely explored, specifically in the context of hypoxia. Using recombinant inhibin and antibodies to the alpha subunit of inhibin, there are novel roles for inhibin as a permeability inducing factor with implications for tumor cell extravasation. Inhibin induced permeability was dependent on ALK1 and endoglin. The VE-cadherin dependent mechanism of permeability is consistent with prior findings on the effects of other TGFβ family members' roles in promoting vascular permeability, specifically BMP6 (64). BMP6 induced vascular permeability was mediated through the Type 1 receptor ALK2 (64), whereas Type 1 receptor ALK1 was expected to be more critical for inhibin induced vascular permeability. Interestingly, inhibin strongly increased the stable interaction between ALK1 and endoglin (FIG. 6c), in line with both endoglin and ALK1 being required for permeability, and endoglin being critical for VE-cadherin internalization. These findings have broad implications for other TGFβ family members that may regulate permeability dependent on Type 1 receptors. The patch/FRAP studies support the current and previous findings (19). Although there are some reports suggesting that inhibin can bind to ALK4 (50), inhibin does not enhance endoglin-ALK4 complex formation but rather weakens it. Endothelial cells such as HMEC-1 express very little ALK4 compared to ALK1 (19), supporting the idea that inhibin acts in endothelial cells preferentially via ALK1 in line with a potential physiological relevance of inhibin-mediated increase in endoglin-ALK1 interactions. However, these findings in endothelial cells do not contradict the current understanding of inhibin's function in non-endothelial cells, which may express more ALK4 than ALK1. These findings also do not allow for a conclusion as to whether the ALK1-endoglin complex, which is enhanced by the binding of inhibin, is signaling or kinase competent, as non-signaling receptor complexes may exist and impact signaling in an indirect manner. Such complexes were previously reported in the context of activin and ALK2 (65-66) and need further examination for inhibins.

Targeting inhibin through shRNA knockdown and antibody treatment was found to be an effective anti-angiogenic strategy leading to reduced vascular permeability increased blood vessel size but fewer number of vessels and a likely more normalized vasculature. Interestingly, in analysis of the angiogenic proteome of HEY tumors, permeability promoting cytokines, EGF, IL-8, and DPP4 were significantly lower in shINHA tumors which were less permeable as compared to shControl tumors. Interestingly, the shINHA tumor cells produced more activin and endoglin compared to shControl tumors. Increased activin fits the profile of the shINHA tumors expressing more anti-angiogenic proteins as activin has been shown to inhibit angiogenesis (7) which could also be a result of decreased inhibinα leading to a shift in the balance to increased dimerization of INHBA/B and thereby activin levels. Similarly, increases in tumor cell endoglin levels in shINHA tumors in vivo may reflect compensatory responses to changes in inhibin expression consistent with recent reports on endoglin expression changes in ovarian cancers (67). Whether these changes impact metastasis and angiogenesis and are directly related to changes in inhibin levels in patients remains to be examined. Which of these altered proteins contributes the most to the either pro or anti-angiogenic tumor microenvironment remains to be determined. In the mouse host cells where inhibin is likely to interact with endoglin from the endothelia to affect angiogenesis, endoglin levels were slightly higher in shControl receiving hosts that had more vessels compared to shINHA. These findings also suggest that blocking inhibin could shift the balance between pro and anti-angiogenic genes.

It is believed that anti-inhibin in a therapeutic regimen can reduce tumor growth in vivo. The subcutaneous model utilized does not induce ascites formation, unlike the intraperitoneal model used previously, where mice with shINHA tumors produced less ascites than those with shControl tumors (19). However, this model was chosen as it better allows for evaluation of the vasculature in vivo and short exposure to hypoxia leads to increased tumor growth as seen here and as seen in other models as well (68, 69). As discussed further herein, inhibin is elevated in patient ascites which supports the idea that inhibin may promote ascites formation, likely through increased vascular permeability. The effectiveness of anti-angiogenic therapies is attributed to increased vascular normalization resulting in reduced intra-tumoral hypoxia, perfused and functional vessels that improve delivery of other chemotherapeutics and enhanced immune response (70). Further studies utilizing intraperitoneal or intrabursal models that present additional steps of disease progression and metastasis are warranted to evaluate hypoxia and anti-inhibin approaches therein. Resistance to current anti-angiogenic therapies is also common and inhibin A levels have been reported to be increased in patients non-responsive to anti-angiogenic therapy (71) (combination of TRC105 and Bevacizumab) indicating inhibin as a potential alternative mechanism of angiogenesis in tumors resistant to other anti-angiogenic therapies. Further studies exploring the impact of anti-inhibin therapy on the effectiveness of chemotherapeutics and anti-tumor immune response as well is most certainly warranted.

In conclusion, the examples discussed in detail below show that targeting inhibin is an effective anti-angiogenic strategy. There is a contextual mechanism for the regulation of inhibin directly driven by hypoxia and HIF-1 and fully define inhibin's contributions to hypoxia induced angiogenesis. Accordingly, and without being bound by theory, it is believed that targeting inhibin may have potential improved therapeutic value in post-menopausal cancers including a significant percentage of ovarian cancers.

### Examples

### Methods and Materials

### Cell Lines and Reagents

Ovarian epithelial carcinoma cell lines were from ATCC, the NCI cell line repository through an MTA, or were as indicated. Cell line authentication was performed at the Heflin Center for Genomic Science Core Laboratories at UAB. HMEC-1s were grown per ATCC instructions. COS7 cells were grown in Dublecco's modified Eagle's medium with 10% FBS, 100 U penicillin/streptomycin and L-glutamine. Mouse embryonic endothelial cells (MEEC) WT and ENG−/− were grown as previously described (47). Epithelial carcinoma cell lines HEY, OVCA420, SKOV3 and PA1 were cultured in RPMI-1640 containing L-glutamine, 10% FBS and 100 U of penicillin-streptomycin (72). OVCAR5 and HEK293 were cultured in DMEM containing 10% FBS and 100 U of penicillin-streptomycin. ID8ip2Luc was a kind gift from Jill Slack-Davis (73) and cultured in DMEM containing 4% FBS, 100 U of penicillin-streptomycin, 5 μg/mL of insulin, 5 μg/mL of transferrin, and 5 ng/mL of sodium selenite. All cell lines were maintained at 37° C. in a humidified incubator at 5% CO₂, routinely checked for myco-plasma and experiments were conducted within 3-6 passages depending on the cell line. For hypoxia experiments, a ProOx Model C21 was used and set to 0.2% O₂ and 5% CO₂. Anti-inhibin PO/23 and R1 antibodies were obtained from Oxford-Brookes university through an MTA and from Biocare Medical. INHA promoter driven luciferase reporter construct was generated through restriction cloning into pGL4.10 luciferase plasmid. Primers were designed to 547 base pairs of the INHA promoter containing the first HRE site with NheI and XhoI restriction sites on the ends. Insert was amplified from PA1 genomic DNA. Insert was ligated into pGL4.10 plasmid with T4 DNA ligase and INHA promoter region was verified through Sanger sequencing.

### Generation of Cell Lines

INHA and ARNT knockdown were generated in HEY cells infected with shRNA lentivirus, followed by selection in 2.5 μg/ml Puromycin and stable cell lines maintained in 1 μg/ml Puromycin. Luc/GFP cell lines were generated using pHIV-Luc-ZsGreen construct. Transient DNA transfections in HEK293 were performed using Lipofectamine 3000. siRNA transfections were performed using RNAiMax. In HEK293 transfections, single siRNA was used while pooled siRNA was used in HEY and OV90 transfections. Lentiviral particles were generated at the Center for Targeted Therapeutics Core Facility at the University of South Carolina. shRNA and siRNA sequences are listed in Table 1.

TABLE 1

| Target | Source | Identifier | Target Sequence |
|---|---|---|---|
| shINHA | Sigma Aldrich | TRCN0000063904 | CCTCGGATGGAGGTTACTCTT |
| shARNT | Sigma Aldrich | TRCN0000003818 | CATTGTCCAGAGGGCTATTAA |
| siHIF-1 | Dharmacon | J-004018-07-0002 | GAACAAAUACAUGGGAUUA |
| siHIF-1 | Dharmacon | J-004018-08-0002 | AGAAUGAAGUGUACCCUA |
| siHIF-2 | Dharmacon | J-004814-06-0002 | GGCAGCACCUCACAUUUGA |
| siHIF-2 | Dharmacon | J-004814-07-0002 | GAGCGCAAAUGUACCCAAU |

### RNA Isolation and RT-qPCR

Total RNA was harvested using Trizol/Chloroform extraction. RNA was transcribed using iScript Reverse Transcription Supermix and iTaq Universal SYBR Green Supermix. Expression data was normalized to RPL13A. qRT-PCR primer sequences are listed in resource Table 2.

TABLE 2

| Primers | Forward | Reverse |
|---|---|---|
| Human | | |
| RPL13A | AGATGGCGGAGGTGCAG | GGCCCAGCAGTACCTGTTTA |
| | (SEQ ID NO: 1) | (SEQ ID NO: 2) |
| INHA | CGCTCAACTCCCCTGATGTC | GGGTACACGATCCACCGTTC |
| | (SEQ ID NO: 3) | (SEQ ID NO: 4) |
| VEGF | CGCCAACCACAACATGCAG | GCTCCACGAAGGATGCCAC |
| | (SEQ ID NO: 5) | (SEQ ID NO: 6) |
| ENG | GCCATCCAATCGAGACCCTG | TGATTGTTGGACTCCTCAGTG |
| | (SEQ ID NO: 7) | (SEQ ID NO: 8) |
| TGFBR3 | CGTCAGGAGGCACACACTTA | CACATTTGACAGACAGGGCAAT |
| | (SEQ ID NO: 9) | (SEQ ID NO: 10) |
| INHBA | GAACGGGTATGTGGAGATAGA | TGTTCCTGACTCGGCAAA |
| | G (SEQ ID NO: 11) | (SEQ ID NO: 12) |
| INHBB | GCGCGTTTCCGAAATCATCA | AGGTTCTGGTTGCCTTCGTT |
| | (SEQ ID NO: 13) | (SEQ ID NO: 14) |
| ARNT | TGACTCCTGTTTTGAACCAGC | CTGCTCACGAAGTTTATCCACAT |
| | (SEQ ID NO: 15) | (SEQ ID NO: 16) |
| Mouse | | |
| RPL13A | CAAGGTTGTTCGGCTGAAGC | GCTGTCACTGCCTGGTACTT |
| | (SEQ ID NO: 17) | (SEQ ID NO: 18) |
| INHA | AGGAAGATGTCTCCCAGGCT | GTTGGGATGGCCGGAATACA |
| | (SEQ ID NO: 19) | (SEQ ID NO: 20) |
| VEGF | ACGACAGAAGGAGAGCAGAAG | ATGTCCACCAGGGTCTCAATC |
| | (SEQ ID NO: 21) | (SEQ ID NO: 22) |
| ChIP | | |
| Primers | | |
| INHA | GGGATGTTCAGGTCCATCAG | CACACTGTAGTTGTGCAGTCAA |
| HRE1 | (SEQ ID NO: 23) | (SEQ ID NO: 24) |
| INHA | CCTCGTTCACCCAGAAGGTC | GATTCCGGCGTCTACGTGTG |
| HRE2 | (SEQ ID NO: 25) | (SEQ ID NO: 26) |

### ELISA

Inhibin ELISA's were performed according to the manufacturer's instructions for the quantitative measurement specifically of total inhibin protein (does not detect activin), that detects inhibin A (dimer of INHA/INHBA), inhibin B (dimer of INHA/INHBB), and free inhibin alpha subunit (INHA), from conditioned media of tumor cells. Cells were grown to 80% confluency in 24 well plates before media was replaced with fresh full serum media. Cells were placed in hypoxia chamber for 24 h and media was collected and concentrated using Amicon Ultra centrifugal filter.

### In Vitro Permeability Assay

In vitro permeability assay was adapted from Martins-Greene 74. 1×10⁵ HMEC-1 cells were plated onto a Matrigel coated 3 μM trans-well filter in full serum media. After 24 h, a second layer of 1×10⁵ HMEC-1 was plated on top to obtain a confluent monolayer of cells. After an additional 24 h, media was replaced with serum free media in the top of the trans-well and either conditioned media (with 2 μg of either R1, PO23, or IgG) or serum free media containing growth factor in the bottom chamber as indicated in legends. FITC-dextran was added to the lower chamber (10 μg/ml). At indicated time points 10 μL aliquots were taken from the top chamber in triplicate and measured using microplate reader for FITC-dextran passage. At end point, filters were stained with crystal violet to confirm equal monolayers were achieved.

### Trans-Well Migration Assay

75,000 HMEC-1 were plated on a fibronectin coated (10 μg/mL) 8 μm trans-well filter in serum free media. Conditioned media (with 2 μg of either R1, PO/23, or IgG) or serum free media containing 1 nM inhibin A or VEGFA was used as a chemoattractant in the bottom chamber. After 24 h, unmigrated cells were scraped off the apical side, migrated cells were fixed in methanol:acetic acid, and nuclei were stained with Hoechst. Three random images were taken per filter using 10× objective on EVOS M7000 microscope. Nuclei were counted using ImageJ.

### Trans-Endothelial Migration Assay

HMEC-1 were grown on 8 μm trans-well filters as per permeability assay. HMEC-1 monolayer was treated with 1 nM inhibin A or untreated for 4 h. After 4 h of treatment, 150 000 HEY-LucGFP expressing cells were plated on top of the HMEC-1 monolayer and allowed to invade for 18 h. Filters were fixed in 4% paraformaldehyde, cells on the apical side of the filter were scraped off, and filters were mounted on glass slides for imaging. Migration of GFP⁺ cells was visualized using 10× objective on EVOS M7000 microscope. Three random fields were captured per filter and GFP⁺ cells were counted using ImageJ software. Thresholding, circularity and size gating were used to exclude unmigrated cells and artifacts.

### Chromatin Immunoprecipitation

Chromatin immunoprecipitation protocol was adapted from ABCAM. Briefly, OV90 or OVCAR5 cells were grown in 150 cm² dishes until 80% confluency was reached. Cells were kept under normoxia or placed in the hypoxia chamber set at 0.2% O₂ for either 12 h (OVCAR5) or 24 h (OV90). DNA was crosslinked using 0.75% formaldehyde and sheared by sonication to fragment sizes between 100-400 bp. DNA was immunoprecipitated with Dyna-beads and either HIF-1ca antibody or Normal Rabbit IgG as a control. DNA was purified using Purelink PCR Purification kit and amplified using RT-qPCR with ChIP primers.

### Luciferase Assay

HEK293 cells were seeded into 24 well plate and co-transfected with a luciferase reporter containing 547 base pairs of the INHA promoter (pGL4.10 INHA) and a SV-40 (Renilla internal control vector). For HIF-1 overexpression, cells were also co-transfected with pcDNA3-HA-HIF1aP402A/P564A or PCDNA3.1. One day after transfection, cells were left in a normoxia incubator or moved to hypoxia chamber (0.2% O₂) for 24 h. Luciferase activity was measured using the Dual Luciferase Reporter Assay System by calculating the ratio between luciferase and Renilla and normalized to normoxia or PCDNA3.1 as indicated in legends.

### Immunofluorescence

HMEC-1 cells were grown to confluence on fibronectin (10 μg/mL) and treated with either 1 nM inhibin A or VEGFA for 30 min in serum free media. Cells were fixed in 4% paraformaldehyde and permeabilized with 0.2% TritonX-100, followed by blocking with 5% BSA in PBS for 1 hr. VE-cadherin was labeled with anti-VE-cadherin antibody overnight at 4° C. followed by AlexaFluor 488 secondary antibody. F-actin was stained with rhodamine-phalloidin and nuclei were labeled with DAPI. Immunofluorescence imaging was performed on EVOS M7000 microscope or Nikon A1 confocal microscope. Actin fibers were quantified by measuring anisotropy using the FibrilTool Plugin in ImageJ (75).

### VE-Cadherin Internalization

HMEC-1 cells grown to confluence on fibronectin (10 μg/mL) coated glass coverslips. Cell surface VE-cadherin was labeled with anti-VE-cadherin antibody at 4° C. for 30 min, washed with ice-cold PBS, and incubated at 37° C. for 30 min with 1 nM inhibin A, 1 nM VEGFA, or serum free media. After internalization was stimulated with growth factor at 37° C., anti-VE-cadherin antibody on the cell surface was removed with mild acid wash. Internalized VE-cadherin was visualized by immunofluorescence microscopy. Internalized VE-cadherin was quantified using BlobFinder software 64⋅76. Nuclei and cytoplasm were delineated and the number of signals per cell was used to quantify internalized VE-cadherin fluorescence.

### Cell Surface Biotinylation

Briefly, MEEC WT or/ENG−/− were grown to confluence on gelatin coated dishes. Cell surface proteins were labeled with 2 mg/mL Sulfo-NH-SS biotin for 30 min at 4° C. After labeling, cells were treated with 1 nM inhibin A or untreated in serum free media for 30 min at 37° C. or left at 4° C. for cell surface control samples. After treatment, cell surface biotin was removed with 20 mM MESNA buffer and internalized biotin labeled protein was isolated with neutravidin resin. Internalized biotin labeled VE-cadherin was detected by Western Blot.

### Epitope-Tagged Plasmids and Transfection of COS7 Cells for Patch/FRAP Studies

The following plasmids were donated by Prof. G. C. Blobe, Duke University Medical Center: HA-tagged endoglin (endoglin-L) in pDisplay, myc-endoglin generated by PCR incorporation of the myc tag sequence into untagged endoglin in pDisplay and re-cloned in pcDNA3.1, and HA- or myc-tagged ALK1 in pcDNA3. 177. Human ALK4 with C-terminal myc-DDK tags in pCMV6 was obtained from OdGene Technologies (Rockville, MD), and subcloned into pcDNA3.1 by PCR followed by restriction digest and re-ligation. A stop codon was introduced at nucleotide 1516 to delete the C-terminal tags to generate untagged ALK4. This was followed by insertion of N-terminal HA tag by overlapping PCR after nucleotide 72 to generate extracellularly tagged HA-ALK4. All constructs were verified by sequencing. COS7 cells were transfected using TransIT-LT1 Mir2300 according to manufacturer's instructions. For Patch/FRAP experiments, cells grown on glass coverslips in 6-wells plates were transfected with different combinations of these vectors encoding myc- and/or HA-tagged receptor constructs. The amounts of the vectors (between 0.5 and 1 μg DNA) were adjusted to yield similar cell surface expression levels, determined by quantitative immunofluorescence.

### Fluorescent Antibody Labeling and IgG-Mediated Cross-Linking for Patch/FRAP

COS7 cells were transfected with various combinations of the above epitope-tagged expression vectors. After 24 h, The cells were serum-starved (1% FBS, 30 min, 37° C.), washed with cold Hank's balanced salt solution (HBSS containing 20 mM HEPES, pH 7.2) and 2% BSA (HBSS/HEPES/BSA), and blocked with normal goat γ-globulin (200 μg/ml, 30 min, 4° C.). For FRAP studies on singly expressed receptors, the cells were then labeled successively at 4° C. in HBSS/HEPES/BSA (45 min incubations) with: (i) monovalent murine Fab′ anti myc tag (αmyc) or anti HA tag (αHA; 40 μg/ml), prepared from the respective IgGs as described by us earlier (78); (ii) Alexa 546-Fab′ goat anti mouse (GαM; 40 μg/ml), prepared from the respective F(ab′)₂ as described (79). For patch/FRAP studies, they were labeled by one of two protocols. Protocol 1 employed successive labeling with: (i) monovalent mouse Fab′ αmyc (40 μg/ml), alone or together with HA. 11 rabbit αHA IgG (20 μg/ml) and (ii) Alexa 546-Fab′ GαM (40 μg/ml) alone or together with Alexa 488-IgG goat anti rabbit (GαR; 20 μg/ml). This protocol results in the HA-tagged receptor crosslinked and immobilized by IgGs, whereas the myc-tagged receptor, whose lateral diffusion is then measured by FRAP, is labeled exclusively by monovalent Fab′. Alternatively, protocol 2 was employed for immobilizing the myc-tagged receptor and measuring the lateral diffusion of a co-expressed Fab′-labeled HA-tagged receptor: (i) monovalent mouse Fab′ αHA (40 μg/ml) together with chicken IgY αmyc (20 μg/ml) and (ii) Cy3-Fab′ donkey anti mouse (DαM; 40 μg/ml) together with FITC-IgG donkey anti chicken (DαC; 20 μg/ml). In experiments with inhibin A, the ligand was added after starvation along with the normal goat γ-globulin and maintained at the same concentration throughout the labeling steps and FRAP measurements.

### FRAP and Patch/FRAP

COS7 cells co-expressing epitope-tagged receptors labeled fluorescently by anti-tag Fab′ fragments as described above were subjected to FRAP or patch/FRAP experiments as described 49. FRAP studies were conducted at 15° C., replacing samples after 20 min to minimize internalization. An argon-ion laser beam (Innova 70 C, Coherent, Santa Clara, CA) was focused through a fluorescence microscope (Axioimager.D1; Carl Zeiss MicroImaging, Jena, Germany) to a Gaussian spot of 0.77±0.03 μm (Planapochromat 63x/1.4 NA oil-immersion objective). After a brief measurement at monitoring intensity (528.7 nm, 1 μW), a 5 mW pulse (20 ms) bleached 60-75% of the fluorescence in the illuminated region, and fluorescence recovery was followed by the monitoring beam. Values of D and R_{f} were extracted from the FRAP curves by nonlinear regression analysis, fitting to a lateral diffusion process (49). Patch/FRAP studies were conducted analogously, except that IgG-mediated cross-linking of epitope-tagged endoglin preceded the measurement (49).

### Patient Ascites

Specimens from patients diagnosed with primary ovarian cancer was collected and banked after informed consent at Duke University Medical Center, with approval for the study from Duke University's institutional research ethics board. ELISA's were conducted using ELISA for Total inhibin from Ansh labs (#AL-134).

### Public Data Mining

Clinical data and normalized RNA-seq were obtained from cBioportal32. The ovarian serous cystadenocarcinoma (TCGA, PanCancer Atlas) and breast invasive carcinoma (TCGA, PanCancer Atlas) were assessed for INHA expression and hypoxia (Buffa or Winter) scores. INHA expression was plotted against hypoxia score for each patient for correlation analysis.

### In Vivo Assays

All animal studies and mouse procedures were conducted in accordance with ethical procedures after approval by UAB's IACUC prior to study commencement.

### Matrigel Plug Assay

Matrigel plugs were formed using 200 μL of Matrigel mixed with 50 μL of HEY conditioned media and injected subcutaneously into the underside of BALB/c female mice aged 5-6 weeks. For conditioned media, HEY cells were grown until 80% confluence in 24 well plate before media was replaced with fresh full serum media. Cells were placed in hypoxia chamber for 24 h and media was collected and concentrated to 50 sL Savant SpeedVac SPD1030. Conditioned media was incubated with 2 μg of either R1 or IgG overnight before injection. Plugs were harvested 12 days after injection and hemoglobin content was determined according to Drabkin's method (19).

### In Vivo Subcutaneous Tumor Growth and Permeability Analysis

3×10⁶ HEY cells either exposed to normoxia or hypoxia (0.2% O₂) for 24 h were subcutaneously injected into right flank of 6-week old Ncr Nude mice (Taconic). Tumor volume ((L×W²)/2) was calculated by caliper measurements every other day starting at day 10 until harvest at day 30. In animals receiving anti-inhibin treatment, R1 (BioCare) was administered IP at 2 mg/kg three times weekly. Da Vinci Green diluent (BioCare) was administered as vehicle.

For measurement of permeability, tumors were harvested between 700-800 mm³. At end point, Rhodamine dextran 70 000 MW was intravenously injected at 2 mg/kg 2 h before euthanasia. Tumors were fixed in 10% NBF and sections were analyzed for rhodamine dextran by immunofluorescence on EVOS M7000. Three sections per tumor were quantified and four images per section were taken. Thresholding was performed in ImageJ and kept constant for all images. ROUT analysis (Q=10%) was performed to test for outliers.

For tumor hypoxia analysis, tumors were harvested at varying sizes between 200-1400 mm³. Pimonidazole (HydroxyProbe) was injected intravenously at 60 mg/kg 1 h before sacrifice. Tumors were fixed in 10% NBF and sections were analyzed for pimonidazole adducts using anti-pimonidazole monoclonal antibody.

### Immunofluorescence on Tissues

Briefly, formalin fixed, paraffin-embedded tissues from subcutaneous tumors were deparaffinized by sequential washing with xylene, 100% ethanol, 90% ethanol, 70% ethanol and distilled water for 10 min each. Antigen retrieval was performed by boiling tissues in sodium citrate buffer (pH 6.0). Blocking was performed with Background Punisher. Primary antibodies, anti-pimonidazole (1:50) and anti-CD-31(1:100), were diluted in Da Vinci Green Diluent and incubated overnight at 4° C. in a humidified chamber followed by AlexaFluor 594 secondary antibody. Nuclei were stained with DAPI. 10× images were acquired on EVOS M7000 microscope.

Quantitation of CD-31 labeled vessel size and number as well as pimonidazole was performed in ImageJ. Images were converted to binary and thresholding mask was applied equally to all images. For CD-31, objects smaller than 25 pixels were removed as were deemed too small to be vessels. For each image, average vessel size (area) and average vessel number was measured. Four images per section and two sections per tumor were used for quantitation. For pimonidazole, a 10× stitched image comprising the whole tumor section was used. The total area covered by signal was acquired and divided by total tumor area to calculate the % hypoxic area for each tumor.

### Angiogenesis Proteome Array

Angiogenesis proteome array was performed according to manufacturer's instruction (R&D Systems, Supplementary Data 2). Briefly, tissues were homogenized in PBS with 1% TritonX-100 and PI cocktail. 200 μg of protein was used per sample (two samples for shControl and shINHA tumors each). Pixel intensity was quantified for each dot using ImageStudio software after background subtraction.

### Statistics and Reproducibility

All data are representative of three independent experiments, unless otherwise described in legends. Statistical analyses were performed using GraphPad Prism 9, with statistical test chosen based on experimental set up and specifically described in the figure legends. Data are expressed as mean±SEM. Difference between two groups was assessed using a two-tailed t-test. Multiple group comparisons were carried by the analysis of variance (ANOVA) using One or Two-way ANOVA followed by appropriate post-hoc tests as indicated in Figure legends.

### Expression and Secretion of Inhibin is Regulated by Hypoxia in Ovarian Cancer Cell Lines

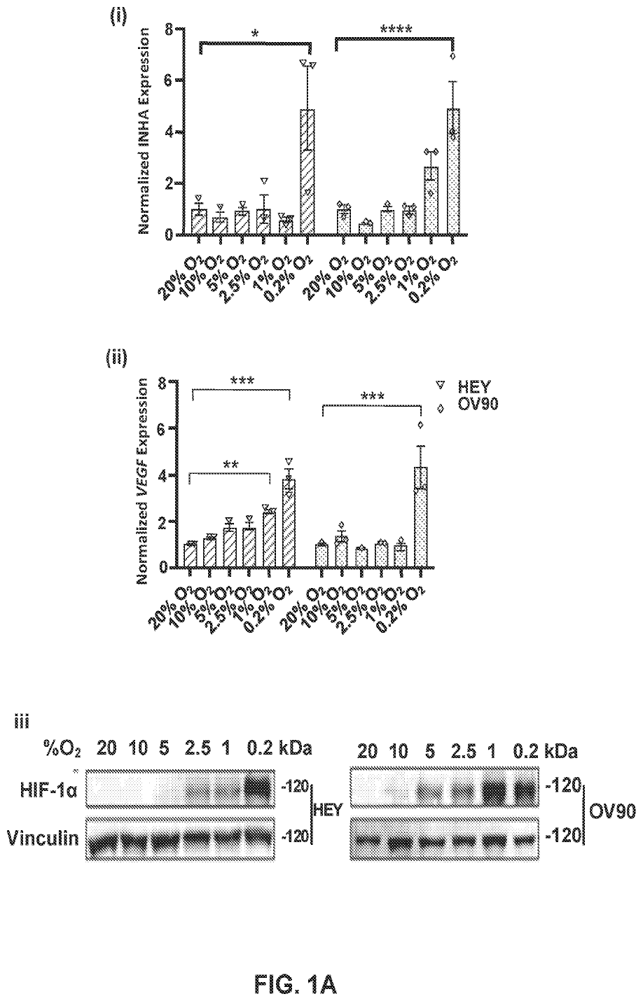

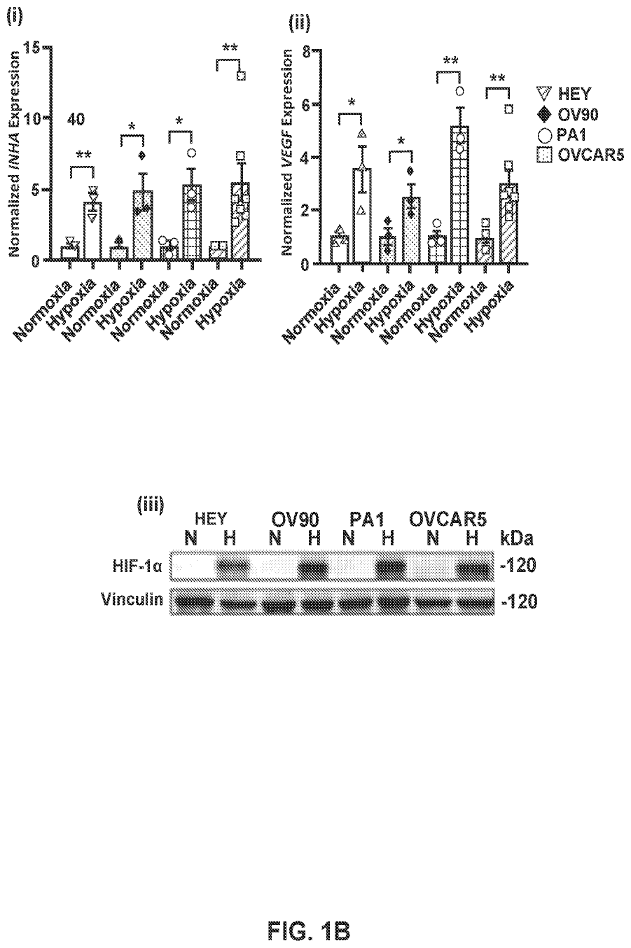

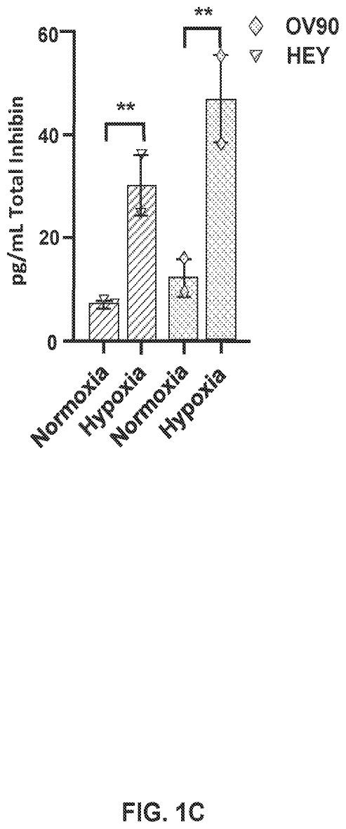

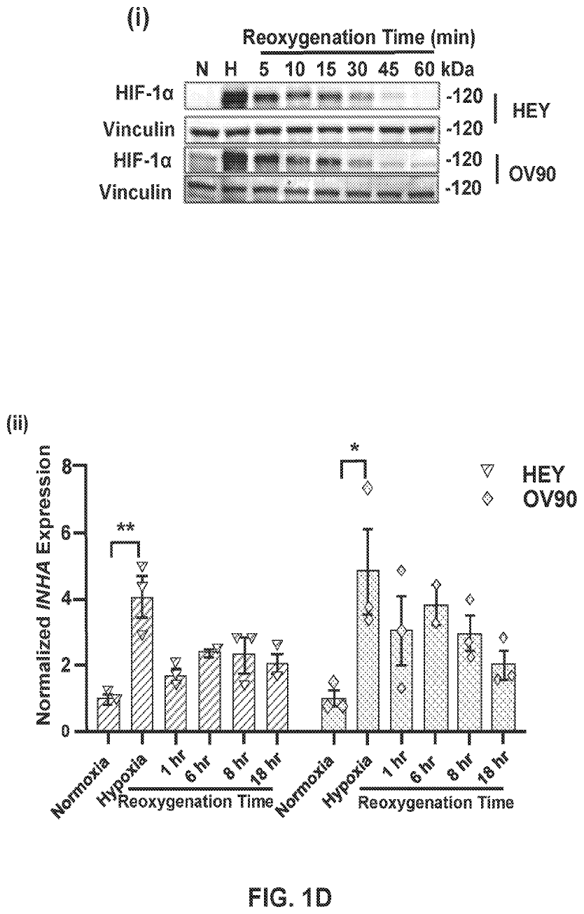

Based on the potential role of inhibins in cancer angiogenesis, the impact of hypoxia, a key regulator of angiogenesis, on INHA expression was tested. The high grade serous ovarian cancer cell lines HEY and OV90 cells were exposed to varying levels of oxygen (control tissue culture conditions (20%), 10%, 5%, 2.5%, 1%, and 0.2% O₂) for 24 h to evaluate INHA expression and VEGFA expression (as a positive control (24)) by semi-quantitative RT-PCR. See FIG. 1. INHA expression was significantly elevated in 0.2% 02 (4.9 times) in both HEY cells and OV90 cells. In OV90 cells, INHA was elevated at 1% (2.7-times) as well, however not significantly. A similar pattern was observed for VEGFA expression with significant increases in both HEY and OV90 at 0.2% O₂ (HEY: 3.8 times and OV90: 4.3 times) and at 1% in HEY (2.4-times). HIF-1 stabilization was evaluated by western blotting to confirm an active hypoxic response that was oxygen tension dependent. To further test the impact of hypoxia on INHA expression, a panel of ovarian cancer cell lines representing a broad spectrum of ovarian cancer subtypes, including HEY, OV90, OVCAR5 of high grade serous origin, and PA1 a teratocarcinoma cell line of the ovary were grown for 12 or 24 h under either hypoxic conditions (0.2% O₂) or normoxic control tissue culture conditions (17-21%). A 3-6 times increase in INHA expression resulted across all four cell lines (HEY: 4-times, OVCAR5: 4.4-times, PA1: 5.28, OV90: 4.8 times. All cell lines showed maximum INHA increases after 24 h of hypoxia growth except for OVCAR5 which increased INHA expression within 12 h under hypoxia. VEGFA was evaluated side by side as a positive control and representative of the hypoxia response in all four cell lines and was elevated 2-6 times (HEY: 3.5 times, OVCAR5: 3.1 times, PA1: 5.18-times, OV90: 2.5 times. A mouse ovarian cancer cell line, ID8ip2, was also tested and INHA expression was elevated 4-times here as well. HIF-1α stabilization in all cell lines confirmed by westerns indicated an active response to hypoxia.

FIG. 1A shows relative qRT-PCR analysis of (i) INHA and (ii) VEGFA mRNA expression normalized to levels in 20% O₂ in HEY and OV90 cells exposed to indicated oxygen concentration for 24 hrs. Mean±SEM, (n=3). *p<0.05; **p<0.01; ***p<0.001; ****p<0.0001, One-way ANOVA followed by Tukey's multiple comparison. (iii) Western blot of HIF-1α stabilization at indicated oxygen concentrations in HEY (left) and OV90 (right). FIG. 1B shows relative qRT-PCR analysis of (i) INHA and (ii) VEGFA mRNA expression normalized to corresponding levels in normoxia in indicated cells grown under hypoxia (0.2%) or normoxia (17-21%) for 24 h except for OVCAR5 (12 h). Mean±SEM, n of independent trials for PA1=3, OVCAR5 i=7, HEY n=3, OV90 i=3, *p<0.05, **p<0.01, unpaired t-test. (iii) Western blot of HIF-1α stabilization in indicated cell lines. FIG. 1C shows total inhibin ELISA (inhibin A/B, inhibinα) of conditioned media collected from OV90 and HEY cells grown in normoxia or after 24 h exposure to hypoxia (0.2% O₂). Mean±SEM, (n=3). **p<0.01, unpaired i-test. FIG. 1D shows (i) Western blot of HIF-1α levels in HEY and OV90 following exposure to hypoxia (0.2% O₂) for 24 h and after indicated reoxygenation times. (ii) Relative qRT-PCR analysis of INHA expression in HEY and OV90 cells following exposure to hypoxia (0.2% O₂) and indicated reoxygenation time normalized to corresponding levels in normoxia. Mean±SEM, (n=3). *p<0.05; **p<0.01, One-way ANOVA followed by Tukey's multiple comparison.

INHA translates into the protein inhibinα which can be secreted as a free monomer or can dimerize with INHBA or INHBB to produce dimeric functional inhibin A or inhibin B12. Thus, total inhibin ELISA (enzyme-linked immunosorbent assay), specific to inhibinα so as to detect all three inhibin forms, was used to test if the changes in INHA mRNA resulted in alterations to secreted protein. Conditioned media collected from HEY and OV90 exposed to hypoxia increased total inhibin protein secretion as well, (4.2-times in HEY and 3.8 times OV90, FIG. 1C). These data suggest that INHA mRNA and functional secreted inhibin protein, is increased by hypoxia.

Since total inhibin protein, reflecting either inhibin A/B and free inhibinα, increased in response to hypoxia (FIG. 1C), mRNA changes were evaluated in INHBA and INHBB subunits in HEY and OV90 cells. While INHA was increased three to five-times in response to hypoxia (FIG. 1B), INHBA and INHBB levels were unchanged in the two cell lines evaluated indicating that changes in inhibin protein levels FIG. 1C) were largely related to increases in inhibinα. The INHA response to hypoxia was also more robust in tumor cells as compared to endothelial cells (HMEC-1) grown under hypoxia (0.2% O₂) for either 12 h or 24 h indicating that inhibinα increases in response to hypoxia occur more significantly in tumor cells.

To test if INHA expression remains elevated after re-exposure to oxygen, the first determination was how long HIF-1 protein remained stabilized in cells when returned to normoxic conditions (reoxygenation) after 24 h exposure to hypoxia. HIF-1 protein began to decrease 5 min after re-exposure to hypoxia (reoxygenation) and went back to baseline at 60 min in HEY and OV90 cells FIG. 1D(i)). Since HIF-1 levels return to baseline after 60 min, time course for testing INHA expression after reoxygenation began at 1 h. In HEY cells, INHA expression was increased four-times upon exposure to hypoxia (FIG. 1D(ii)). Upon 1 h of reoxygenation, INHA expression decreased significantly in both cell lines and was no longer statistically different from normoxia grown cells (FIG. 1D(ii)). Slight elevation in INHA levels remained, particularly in OV90 cells for the duration of the time course (FIG. 1D(ii)) that did not however reach statistical significance. Taken together, these data strongly indicate that inhibinα mRNA and protein expression is increased under hypoxia conditions. Inhibinα is increased in ovarian cancer spheroids, patients, and tumor xenografts.

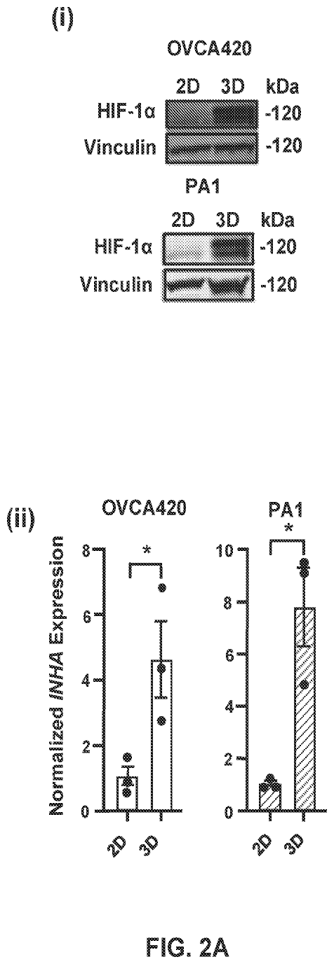

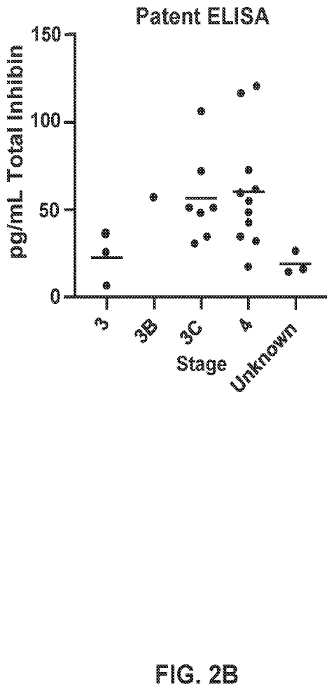

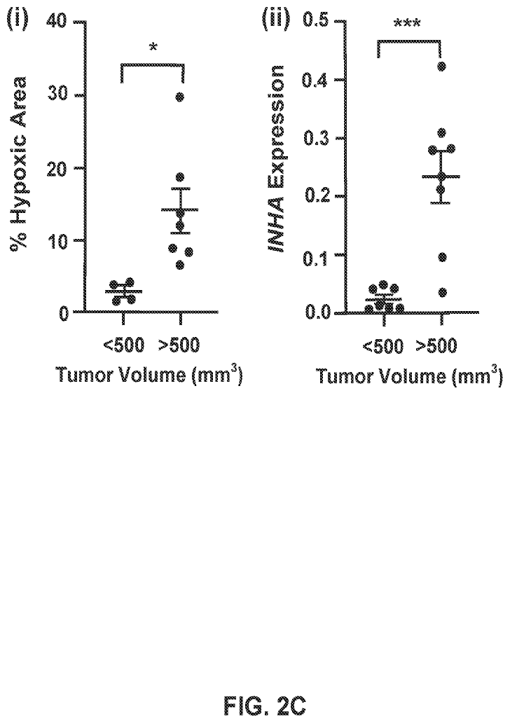

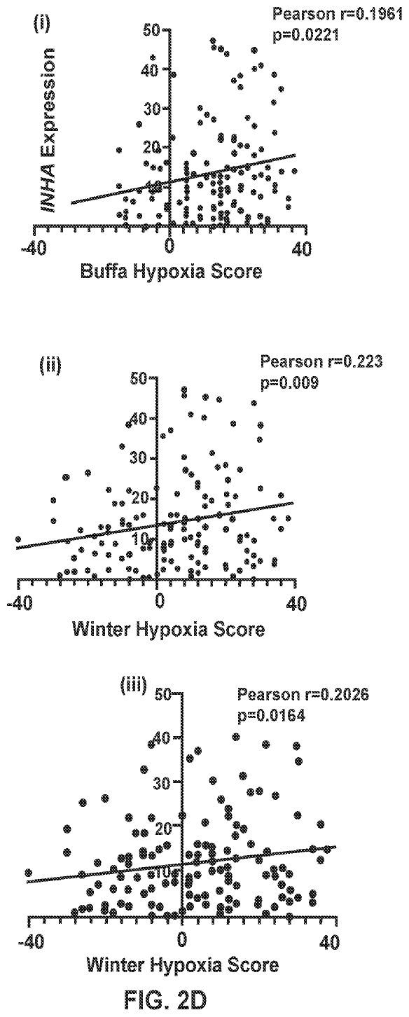

To evaluate other pathologically relevant hypoxic conditions pertinent to ovarian cancer growth and metastasis, hypoxia and INHA expression in cells grown in spheroids under anchorage independence were investigated, an environment that is often hypoxic (25). PA1 and OVCA420 cells were chosen due to their ability to form spheroids (26-27). Cells were grown on poly-hema coated plates for either 72 h (PA1) or 48 h (OVCA420). Under such anchorage independent conditions (referred to as 3D), where HIF-1α was stabilized (FIG. 2A(i)), INHA was increased 7.8 times in PA1 and 4.6 times in OVCA420 when compared to 2D growth conditions in a dish (FIG. 2A(ii)).

FIG. 2A shows (i) Western blotting of HIF-1α protein from indicated cells grown in either 2D or under anchorage independence (3D) conditions, vinculin is loading control and (ii) relative qRT-PCR of INHA mRNA expression in OVCA420 and PA1 after 48 h (OVCA420) or 72 h (PA1) of growth under anchorage independence (3D). Mean±SEM, n=3. **p<0.01; ***p<:0.001, unpaired t-test. FIG. 2B shows total inhibin ELISA of ascites fluid from 25 ovarian cancer patients sorted by stage. FIG. 2C shows (i) percent hypoxic area in tumors of indicated size range determined by quantitation of pimonidazole staining in tumors. Graph represents average hypoxic area of all HEY xenograft tumors sorted by size as <500 mm³ or >500 mm³. Mean±SEM, n=4 for <500 mm³ and n=7 for >500 mm³. *p<0.05, unpaired t-test. (ii) Relative qRT-PCR of INHA expression in tumors from indicated sizes of HEY cells implanted subcutaneously. Mean±SEM, n=8. ***p<0.001, unpaired t-test. FIG. 2D shows a correlation analysis between INHA expression and either (i) Buffa or (ii) Winter hypoxia scores from TCGA OVCA (i-ii) or breast (iii) cancer patient data sets from cBioportal measured by RNA-Seq. Correlation analysis was performed by Pearson correlation.

Previous studies have established that in healthy premenopausal women, inhibin levels cycle across the menstrual cycle reaching a peak of 65.6 pg/mL, while in post-menopausal women, total serum inhibin levels are below 5 pg/mL (28). Ovarian cancer patients are commonly post-menopausal (29) and tumor tissues can display higher inhibin levels (19). Whether the peritoneal ascites fluid of advanced ovarian cancer patients, which has been shown to be a hypoxic environment (30) and contains disseminated ovarian cancer spheroids (22), also displays detectable or elevated inhibin levels was assessed. To test if inhibin protein is secreted and detectable in clinical ascites, total inhibin ELISA was performed on a cohort of 25 patient ascites. Total inhibin levels were in the range of 6.7 to 120.53 pg/mL in the ascites fluid, indicating the presence of inhibin protein in ascites fluid (FIG. 2B).

Whether INHA expression was elevated in vivo with increasing xenograft tumor size was then investigated. 5 million HEY cells were subcutaneously implanted and harvested at varying tumor sizes. Tumors greater than 500 mm³ were found to be hypoxic based on pimonidazole staining which has a detection threshold of below 10 mmHg 02, or 1.2% O₂ (4.8 times, FIG. 2C(i) (31). INHA expression was increased 9.8 times in tumors greater than 500 mm³ as compared to tumors less than 500 mm³ (FIG. 2C(ii)). INHA expression was also significantly correlated with tumor size. To further examine the potential clinical relevance of inhibinα expression in response to hypoxia, the TCGA/PanCancer Atlas patient data set from cBioportal (32-33) was analyzed and hypoxia scores were obtained from two different hypoxia gene signatures (Buffa and Winter) (34-35). The signatures consisted of 51 (Buffa) and 99 (Winter) hypoxia related genes from a large meta-analysis of breast and head and neck squamous cell cancer that were independently verified for prognostic value (34-35). Using these signatures, inhibinα (INHA) expression was significantly correlated with both hypoxia Buffa (r=0.1961, p=0.0221) and Winter hypoxia (r=0.223, p=0.009) scores in the ovarian cancer data set (FIGS. 2D(i)-(ii)). Analysis of breast cancer data revealed a similar trend as INHA expression was significantly correlated (r=0.2026, p=0.0165) with the Winter hypoxia score (FIG. 2D(iii)). Taken together, these data strongly indicate that inhibinα mRNA and protein expression are increased under hypoxia conditions in ovarian cancer cell lines, xenograft tumors and in patients. INHA is a direct HIF-1 target under hypoxia.

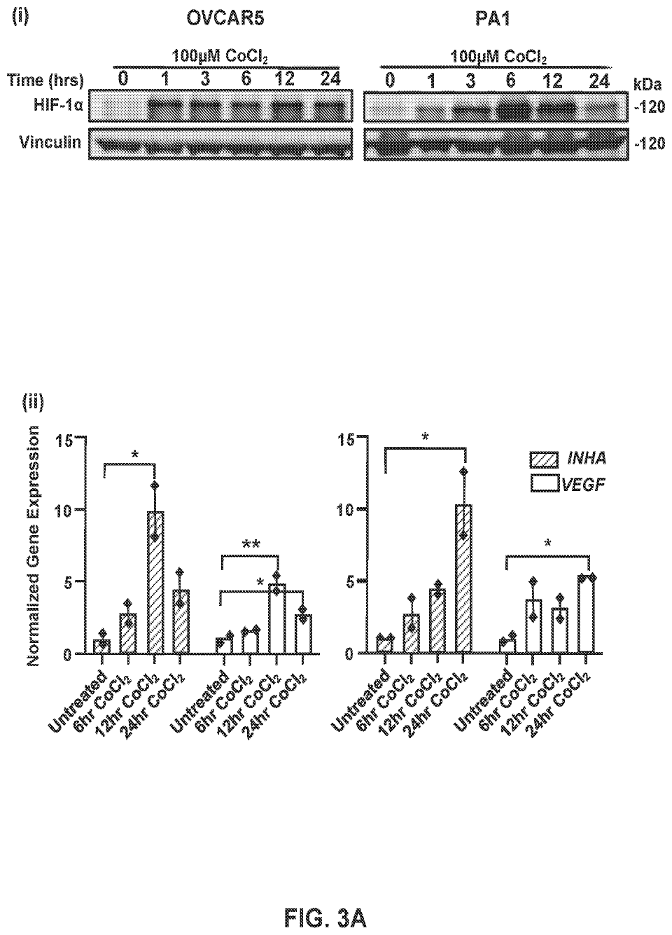

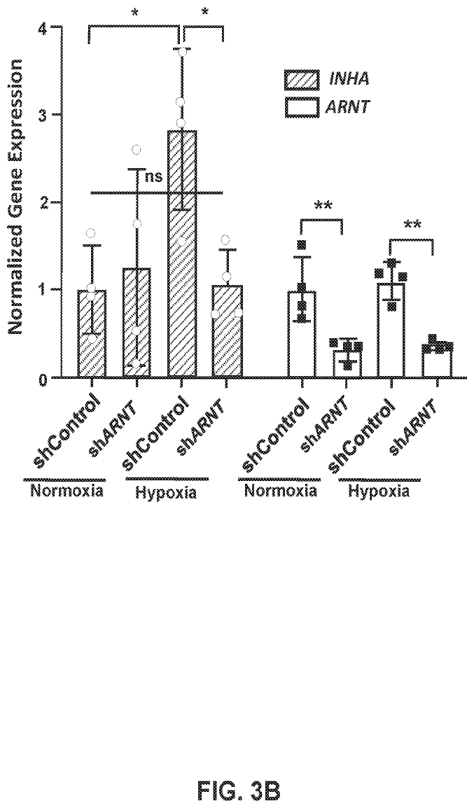

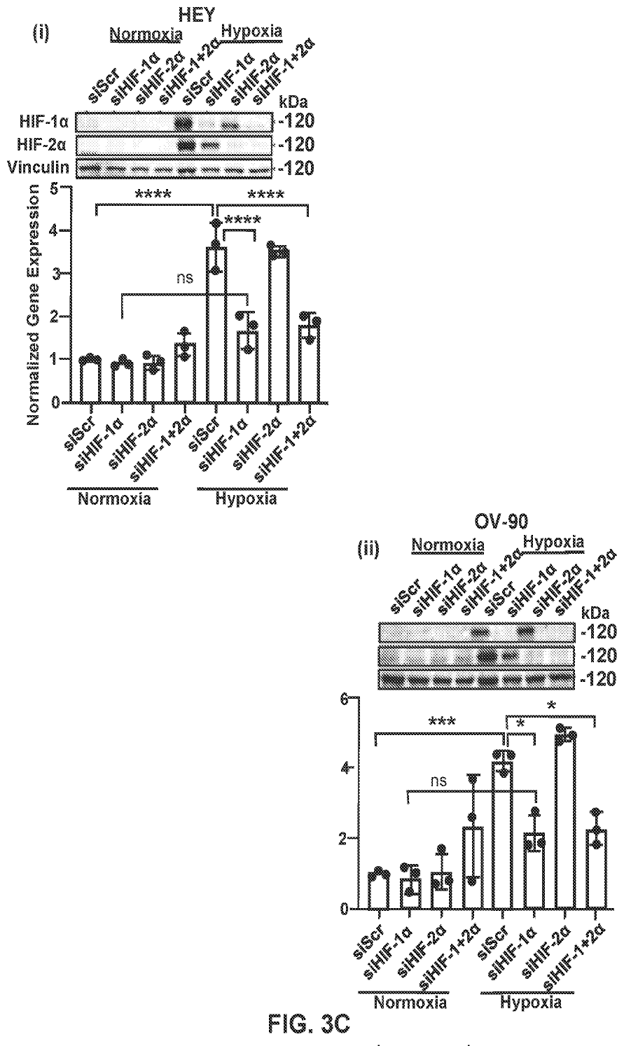

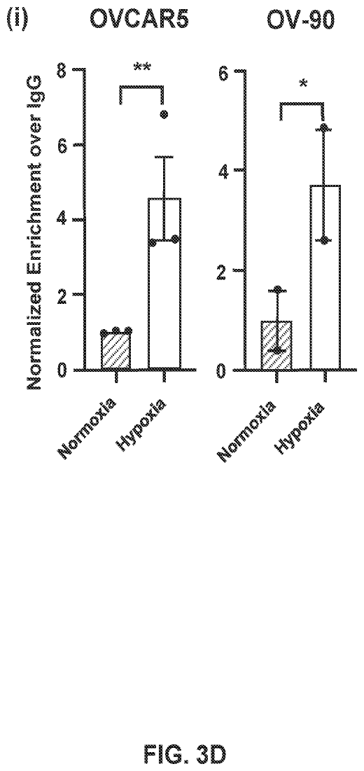

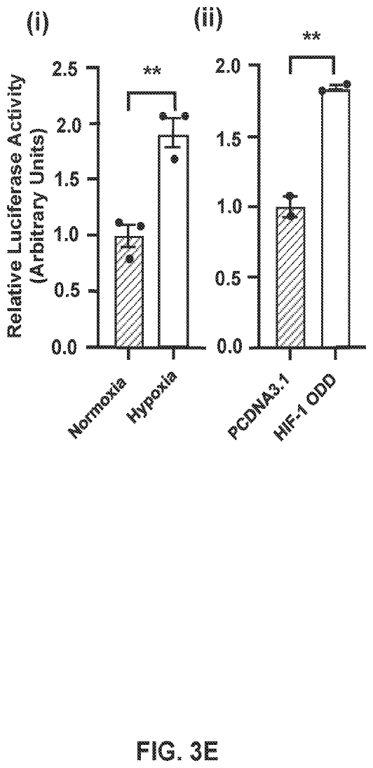

Hypoxia inducible factors (HIFs) are key transcriptional regulators of the hypoxia adaptive response and increase expression of critical pro-angiogenic genes (21). To test whether HIF proteins are regulators of INHA expression, cobalt chloride (CoCl₂), a well characterized chemical stabilizer of HIF's (36), was utilized. HIF-1α was stabilized in PA1 and OVCAR5 cells treated with 100 μM of COCl₂ for either 6, 12, or 24 h (FIG. 3A(i)). INHA expression was significantly increased; 10-times in OVCAR5 after 12 h and 11.5 times in PA1 cells after 24 h of COCl₂ treatment (FIG. 3A(ii)). Maximum increases in INHA expression with CoCl₂ occurred at the same time points as exposure to hypoxia (12 h for OVCAR5 and 24 h for PA1, FIG. 1B(i)). VEGFA, used as a positive control increased 4.8 and 4.3 times at 12 h and 2.7 and 3.7-times at 24 h in both OVCAR5 and PA1, respectively (FIG. 3A(ii)). To test if INHA could be a direct hypoxia target leading to increased inhibinα expression, the effect of reducing the levels of HIF-1β/ARNT, which is the binding partner for all HIF's (37), was evaluated. Stable ARNT knockdown cells were generated in HEY cells (Methods). Control HEY cells increased INHA levels 2.8 times under 0.2% O₂ (FIG. 3B). However, shRNA ARNT lead to a 2.7-times reduction in hypoxia induced increase in INHA mRNA levels (FIG. 3B indicating direct contributions of HIFs' to the regulation of inhibin.

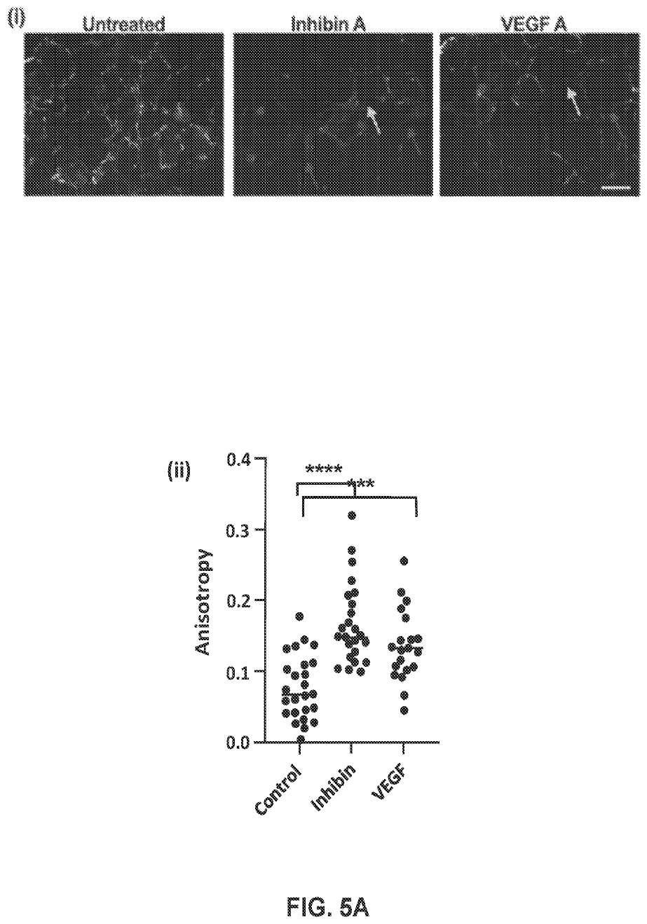

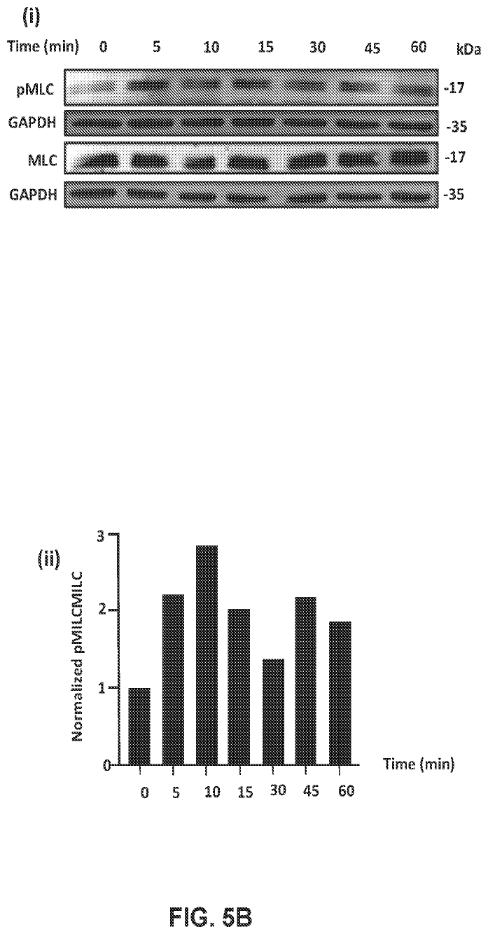

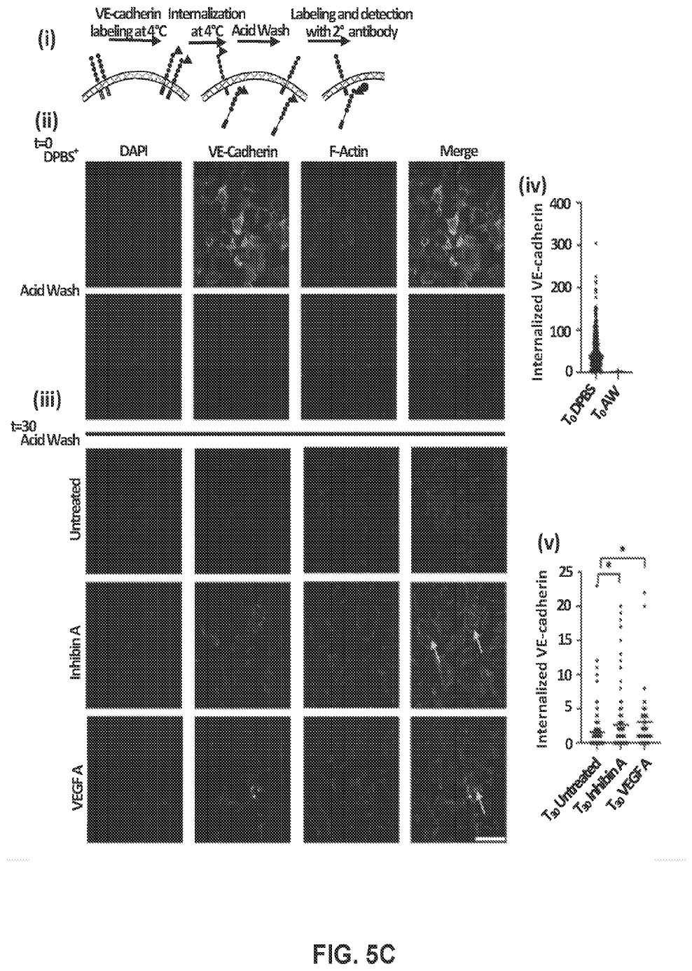

FIG. 3A shows (i) Western blot of HIF-1α at indicated time points after treatment with 100 μM CoCl₂. (ii) Relative qRT-PCR analysis of INHA and VEGF mRNA in OVCAR5 and PA1 cells after indicated time of treatment with 100 μM of CoCl₂ normalized to untreated. Mean±SEM, (n=2). *p<0.05; **p<0.01, One-way ANOVA followed by Tukey's multiple comparison. FIG. 3B shows relative qRT-PCR analysis of INHA and ARNT mRNA in HEY shControl or shARNT cell lines after exposure to hypoxia (0.2% O₂) for 24 h normalized to corresponding shControl normoxia levels. Mean±SEM, (in =4). n.s., not significant; *p<0.05; **p<0.01, unpaired 1-test. FIG. 3C shows representative western blot (above) and relative qRT-PCR analysis of INHA expression (below) from (i) HEY or (ii) OV90 cells transfected with either siScr, siHIF-1α, siHIF-2α, or a combination of siHIF-1/2α and exposed to hypoxia (0.2% O₂) for 24 h. Mean±SEM, (i=3) *p<0.05; ***p<0.001; ****p<0.0001, One-way ANOVA followed by Tukey's multiple comparison test. FIG. 3D shows relative qRT-PCR analysis using primers that amplify the proximal HRE region in the INHA promoter after chromatin immunoprecipitation (ChIP) of HIF-1α in OVCAR5 and OV90 cells. ChIP qRT-PCR results were quantified as normalized enrichment over IgG and normalized to normoxia. Mean±SEM, OVCAR5 (n=3), OV90 (n=2). n.s., not significant; *p<0.05; **p<0.01, Two-way ANOVA followed by Fishers LSD test. FIG. 5E shows Luciferase activity of HEK293 cells transfected with the INHA promoter driven luciferase reporter construct (pGL4.10) and a SV-40 renilla control vector. Cells were either (i) exposed to hypoxia (0.2% O₂) or (ii) co-transfected with HIF-1α overexpression plasmid (HIF-1 ODD) and luciferase activity measured and normalized to either normoxia in (i) or PCDNA3.1 in (ii). Mean±SEM, n=3 (Hypoxia), n=2 (HIF-1 ODD) *p<0.05; **p<0.01, unpaired t-test.

To determine the roles of the HIF-1 and HIF-2 heterodimeric transcriptions factors, that both require ARNT (37), in the transcriptional regulation of INHA siRNA was used to knockdown the levels of HIF-1α and HIF-2a in two ovarian cancer cell lines (OV90 and HEY). Knockdown of HIF-1α and HIF-2a using siRNAs to each isoform individually or a combination of siRNAs was confirmed through western blotting (FIGS. 3C(i) and (ii)). Knockdown of HIF-1α decreased hypoxia induced INHA expression 2.1 times in HEY and 1.9 times in OV90 compared to siScr (siHIF-1α; FIGS. 3C(i) and (ii)). siRNA to HIF-2a did not result in a significant change in hypoxia induced INHA expression compared to control (siHIF-2α; FIGS. 3C(i) and (ii)). To further test that HIF-1 was the predominant HIF isoform required for hypoxia induced INHA expression, a double knockdown of HIF-1α and HIF-2α was utilized. In HEY and OV90 cells the double knockdown resulted in a 2-times and 1.8 times decrease in INHA expression compared to siScr, respectively (siHIF-1,2α; FIG. 3C(i) and (ii)). These data suggest that increases in INHA under hypoxia were more significantly impacted by HIF-1 as compared to HIF-2.

In silico analysis of the INHA gene, which is located at Chr:2q35 revealed two hypoxia response element (HRE) consensus sites within 2 Kb of the promoter, GGCGTGG and CGCGTGG, at −144 and −1789 bp from the transcription start site (TSS)) respectively. These HRE sites conform precisely to the (G/C/T)(A/G)CGTG(G/C) consensus sequence (37). Two hypoxia ancillary sequences (HAS) (CAGGG and CACGG) were also found directly flanking the proximal HRE sequence at −169 and −173 bp from the TSS, respectively. One HAS sequence (CACGT) was found flanking the distal HRE sequence at −1761 bp from TSS. A previously well characterized CREB binding site (CRE) is designated for reference.

To test direct interactions between HIF-1 and the INHA promoter, chromatin immunoprecipitation (ChIP) was performed using OVCAR5 and OV90 cells. Primers were designed to amplify the region including the HRE site closest to the transcription start site (HRE 1) and chromatin shear size optimized accordingly (Methods). Exposure to hypoxia led to a 4-times increase in enrichment of HIF-1 binding to INHA's HRE site in OVCAR5 and 3 times in OV90 (FIG. 3D). The second HRE site is GC rich which led to modest amplification. Despite this, a 2-times increase in HIF-1 enrichment at this site in OV90 cells was observed which was however not statistically significant.

Given the poor enrichment of HIF-1 at the distal promoter site whether the proximal promoter was sufficient to increase INHA levels under hypoxia was evaluated, and if this was dependent on HIF-1. To achieve this, a INHA promoter driven luciferase reporter construct, containing 547 base pairs of the INHA promoter, including the first HRE site (FIG. 3E) was made. The effect of HIF-1 on INHA promoter activity, was evaluated in HEK293 cells exposed to hypoxia (0.2% O₂) for 24 h and compared to cells under normoxia (FIG. 3E(i)), or in the presence or absence of HIF-1 ODD (pcDNA3-HA-HIF1aP402A/P564A) (FIG. 3E(ii)) that prevents degradation of the HIF-1α subunit (38). In un-transfected or control vector expressing cells (pcDNA3.1), INHA promoter driven luciferase activity is increased two times in response to hypoxia (FIG. 3E(i)) that was mimicked by stabilization of HIF-1α (HIF-1 ODD) under normoxia conditions (FIG. 3E(ii)). Further, the requirement of HIF-1α in HEK293 using either control or HIF-1/2α siRNAs was confirmed. HEK293 were exposed to hypoxia for 24 h and efficacy of HIF-1/2α knockdown was confirmed by immunoblotting. Notably, siRNA to HIF-1α (siHIF-1α) decreased hypoxia induced INHA expression 1.8 times as compared to scramble controls (siScr). However, siRNA to HIF-2a resulted in a smaller (1.25-times) and non-significant reduction in INHA expression compared to siScr when exposed to hypoxia. These data point to a central role for HIF-1 in regulating INHA expression under hypoxia.

INHA has been previously reported to be regulated by other factors particularly the cAMP response element binding (CREB) family member in multiple systems (8). The CREB family of transcription factors can act downstream of the hypoxia response (39). To thus test whether cAMP was involved in regulating INHA expression under hypoxia, forskolin (Fsk), an activator of cAMP previously shown to induce INHA expression and the PKA inhibitor, H89, previously shown to inhibit forskolin induced INHA expression (40), was utilized. Treatment of ID8ip2 cells with Fsk increased INHA expression 5.2-times under hypoxia compared to just 2-times under normoxia. This relationship appeared to be additive and not synergistic as addition of the PKA inhibitor, H89, was not able to reduce hypoxia induced INHA expression (Supplementary FIG. 3D(i)). The effect of blocking PKA signaling under hypoxia was also tested in the ovarian cancer cell line OV90. While hypoxia increased INHA expression 4.5 times in OV90 cells, treatment with H89 did not significantly reduce INHA expression under hypoxia (Supplementary FIG. 3D(ii)). Taken together, these data implicate HIF-1 as being the key transcriptional factor responsible for increase of INHA in hypoxia.

### Inhibin Promotes Hypoxia Induced Angiogenesis and Stimulates Endothelial Cell Migration and Vascular Permeability

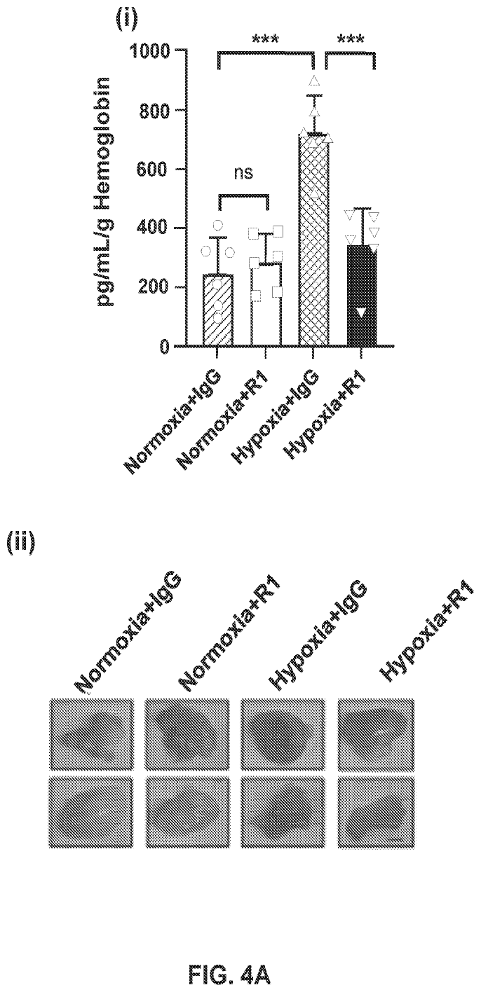

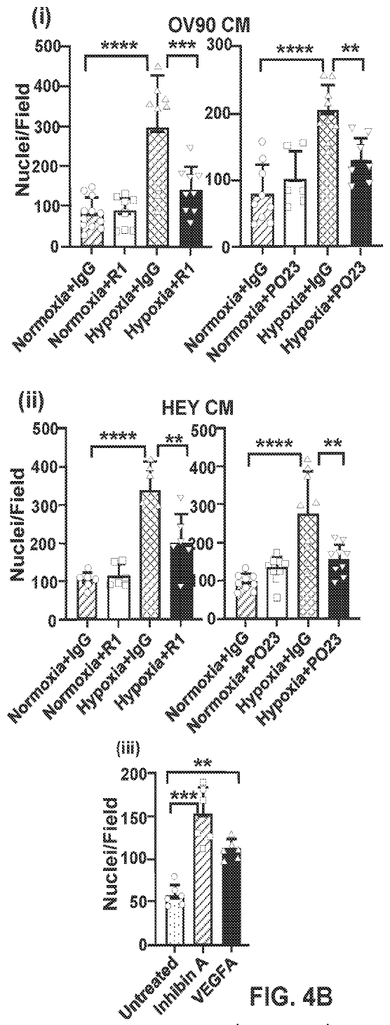

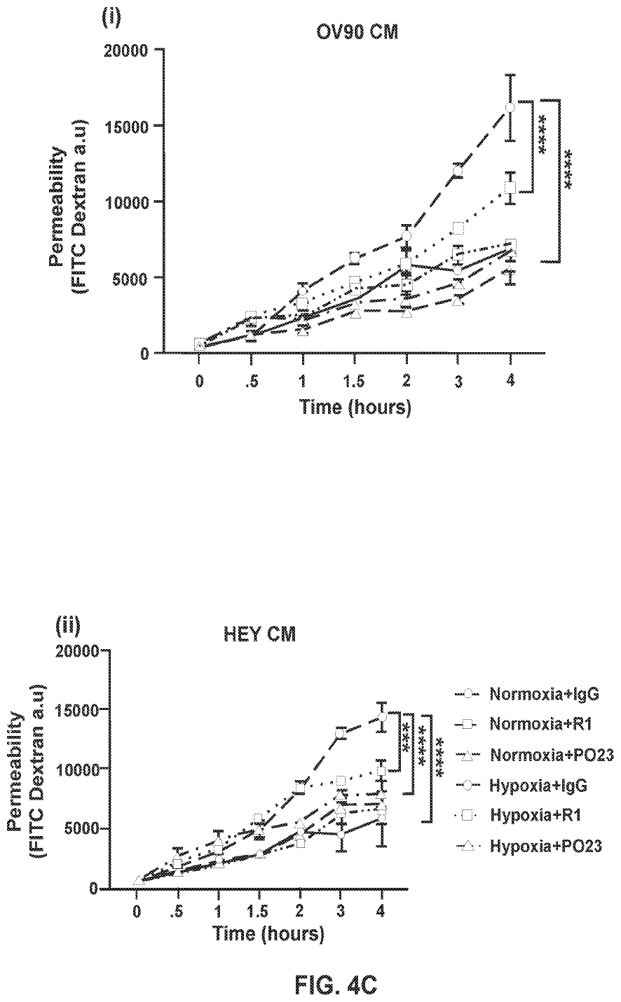

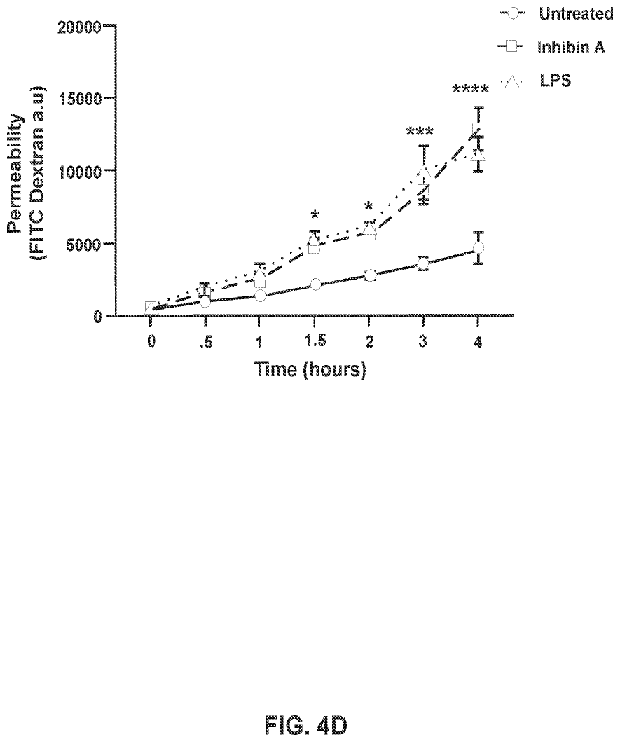

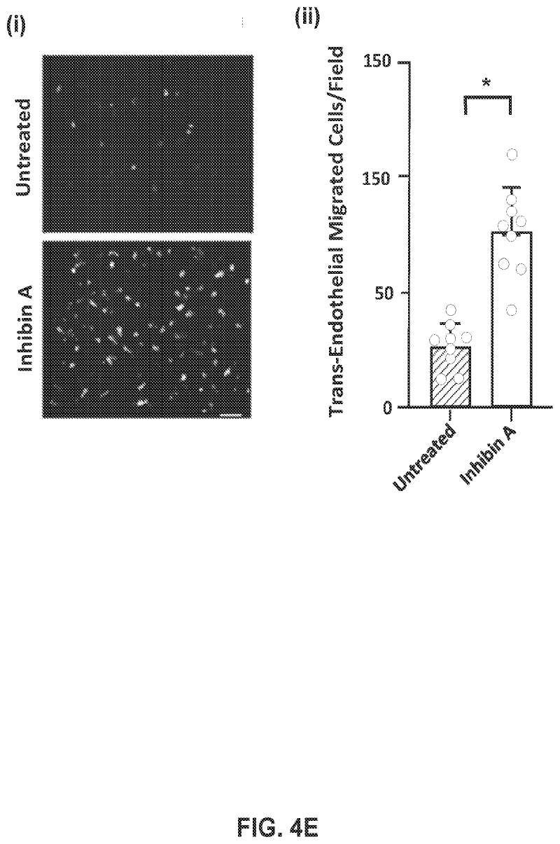

Hypoxia is a key driver of endothelial cell migration and blood vessel permeability within the tumor leading to alterations in angiogenesis (24). To determine the overall contribution of inhibin to hypoxia induced angiogenesis in vivo, an in vivo Matrigel plug assay was utilized. Conditioned media (CM) from HEY tumor cells exposed to normoxia or hypoxia was used to stimulate angiogenesis into the plugs, and a well-established anti-inhibinα antibody, R1 (recognizing the junction between the αN region, and αC region) (41) was used to block inhibin in the CM with IgG as a control. CM from hypoxia grown cells increased hemoglobin in the plugs 2.9 times compared to CM from normoxia grown cells (FIGS. 4A(i) and (ii)). Anti-inhibinα in the hypoxic CM fully reduced the hemoglobin content in the plug (2.1 times suppression, FIG. 4A(i)-(ii)) indicating that inhibin is required for hypoxia induced blood vessel formation in vivo.

FIG. 4A shows (i) hemoglobin content in Matrigel plugs collected 12 days after subcutaneous injection of HEY conditioned media collected from cells exposed to normoxia or hypoxia for 24 h and mixed with either 2 μg of IgG or anti-inhibin R1 antibody. Mean±SEM, i=6 plugs per condition. n.s., not significant; ***p<0.001, One-way ANOVA followed by Tukey's multiple comparison test. (ii) Representative images of Matrigel plugs from (i) Scale bar: 2 mm. FIG. 4B shows quantitation of HMEC-1 migration through fibronectin coated 8 μm trans-well filter (i-ii) towards conditioned media from OV90 or HEY cells exposed to hypoxia (0.2% O₂) with either 2 μg of R1 or PO23 anti-inhibin antibody or IgG as a control, or towards (iii) serum free media containing 1 nM inhibin A or 1 nM VEGFA. Nuclei from three representative fields per filter were counted. Mean±SD. **p<0.01; ***p<0.001; ****p<0.0001, One-way ANOVA followed by Tukey's multiple comparison. FIGS. 4C and D show quantitation of endothelial cell permeability by measuring FITC-dextran changes across a HMEC-1 monolayer treated with (i-ii) conditioned media from (i) OV90 or (ii) HEY cells exposed to hypoxia (0.2% O₂) with either 2 μg of R1 or PO23 anti-inhibin antibody or IgG as a control, or (d) treated with 1 nM inhibin A or 10 μg/mL LPS. Mean±SEM *p<0.05; ***p<0.001; ****p<0.0001, One-way ANOVA followed by Tukey's multiple comparison. FIG. 4E shows HEY trans-endothelial migration (TEM) across HMEC-1 monolayer either treated with inhibin A for 4 h or untreated. (i) Representative transmigrated GFP positive HEY cells and (ii) quantitation of transmigration (n=3). *p<0.05, unpaired 1-test. Scale bar: 100 μm.

Since blood vessel flow is an indication of endothelial cell functionality (42), efforts were made to define the specific effects of increased inhibinα on hypoxia induced endothelial cell biology, specifically endothelial cell chemotaxis and vascular permeability. To determine the impact on endothelial chemotaxis to hypoxic CM, CM from either hypoxia (24 h, 0.2% O₂) or normoxia grown OV90 or HEY cells were used as a chemoattractant to measure migration of human microvascular endothelial cells (HMEC-1; FIG. 4B). Two anti-inhibinα antibodies, R1 and a second well-established antibody PO23 (recognizing the C-terminus of the αC region) (41), were used with IgG controls to test the effect of blocking/sequestering hypoxia produced inhibinα. CM from hypoxia grown tumor cells significantly increased migration of endothelial cells (IgG, FIG. 4B) and incubation of hypoxic CM with anti-inhibinα R1 significantly suppressed hypoxia induced endothelial migration (2.1 and 1.6 times for OV90 and HEY conditioned media respectively, FIGS. 4B(i)-(ii)). Anti-inhibinα PO23 was also able to significantly suppress CM stimulated endothelial migration (1.5 and 1.75-times for OV90 and HEY CM, respectively, FIGS. 4B(i)-(ii)). Similar to the effects of hypoxic CM, recombinant inhibin A was also able to stimulate HMEC-1 migration to similar extents as VEGFA at equimolar amounts (FIG. 4B(iii)).

The effect of CM from hypoxic tumor cells on changes to permeability across an endothelial monolayer using a trans-well permeability assay that measures solute (FITC-dextran) flux across endothelial monolayers was then evaluated. Permeability was monitored across a 4-h time course and CM from hypoxic tumor cells was used to induce permeability across the HMEC-1 monolayer. Effect of inhibin in the CM was evaluated either in the presence of anti-inhibinα (PO23 and R1) or IgG control (FIGS. 4C(i)-(ii)). Both inhibinα antibodies (R1 and PO23) significantly decreased solute flux induced by hypoxic CM from two tumor cell lines, albeit with moderate differences in the kinetics and time to inhibition (FIGS. 4C(i)-(ii)). Specifically, significant inhibition of permeability was seen beginning at 2 h for CM treated with PO23 and 3 h for R1. PO23 was moderately more effective than R1 as it effectively reduced permeability within 1 h (FIGS. 4C(i)-(ii)). Recombinant inhibin was also able to induce endothelial cell permeability to similar extents as lipopolysaccharide (LPS) (FIG. 4D), an established permeability inducing factor (30). Since perturbations to the endothelial barrier are critical to invasion and extravasation of cancer cells during metastasis (43), whether inhibin induced vascular permeability facilitates tumor cell extravasation was tested. To test this, a trans-endothelial cell migration assay was used to mimic the process. HEY tumor cells infected with GFP adenovirus to distinguish them from migrated non-GFP endothelial cells were plated on top of a non-GFP endothelial cell monolayer that was then either pre-treated with 1 nM inhibin A for 4 h or left untreated. HEY GFP tumor cells were 2.9 times more invasive across the inhibin treated monolayer than untreated conditions (FIGS. 4E(i)-(ii)). All together, these data implicate inhibin as a robust contributor to hypoxia mediated angiogenesis, vascular permeability and thereby tumor cell extravasation across the vascular endothelium.

### Inhibin Promotes Vascular Permeability Through Increased VE-Cadherin Trafficking

Endothelial permeability is regulated through changes in junctional proteins which are maintained through contacts with the actin cytoskeleton (44). VE-cadherin is a critical junctional protein involved in regulating endothelial cell permeability (44). To delineate the mechanism of inhibin's effects on vascular permeability, the effect of inhibin on endothelial cell junctions and the actin cytoskeleton through immunofluorescent staining of VE-cadherin and actin (FIG. 5A) were first evaluated. Examination of the actin cytoskeleton revealed contractile actin staining, with a quantifiably significant increase in stress fiber formation after 30 min of inhibin A treatment (two times increase, FIGS. 5A(i)-(ii)). VEGFA treatment was used as a comparison that also led to similar changes in actin stress fiber formation (FIG. 5A). VE-cadherin localization also appeared to be reduced qualitatively at the cell-cell junctions after 30 min of inhibin treatment as compared to untreated cells, suggestive of perturbation of the endothelial cell barrier at the level of the cytoskeleton (FIG. 5A). Loss of VE-cadherin at the cell junctions was also observed in VEGFA treated cells (FIG. 5A). However, total VE-cadherin levels were unchanged in response to inhibin as evaluated over a time course of 60 min indicating no change in the total pool of VE-cadherin in response to inhibin A. Actin contractility and stress fiber assembly is regulated through phosphorylation of myosin light chain (MLC) (440. In accordance, phosphorylation of MLC-2 (Ser19) increased within 5 min of inhibin A treatment and was sustained across a 60-min time course (FIGS. 5B(i)-(ii). Based on the qualitative changes in VE-cadherin in response to inhibin A treatment (FIG. 5A), whether alterations in VE-cadherin at the cell-cell junctions were due to inhibin induced VE-cadherin internalization (FIG. 5C) was tested. To determine this, HMEC-1 membrane localized VE-cadherin was labeled at 4° C. with an anti-VE-cadherin antibody recognizing the extracellular domain. HMEC-1 cells were washed with acid to remove membrane bound anti-VE-cadherin leaving only any internalized VE-cadherin that may have been labeled at 4° C. prior to treatment with inhibin A or VEGFA (FIG. 5C(i)). Stripping of cell surface VE-cadherin was verified by cell surface immunostaining of VE-cadherin with little to no internalized VE-cadherin detected (FIGS. 5C(ii), (iv)). Cells were then either left untreated or treated for 30 min with inhibin A at 37° C. and VE-cadherin evaluated by immunofluorescence (FIG. 5C(iii)). Inhibin A increased the internalized VE-cadherin pool compared to untreated cells 1.4-times (FIG. 5C(v)) and to similar extents as VEGFA (1.6 times, FIG. 5C(v)). The untreated HMEC-1 had 5% of cells with internalized VE-cadherin while inhibin A and VEGFA treated HMEC-1 resulted in 54% and 42%, of cells respectively, with detectable internalized VE-cadherin. These results indicate that inhibin induces rapid changes in the actin cytoskeleton and trafficking of VE-cadherin from the cell junctions of endothelial cells.

FIG. 5A shows (i) representative immunofluorescence images of F-actin (red) or VE-Cadherin (green) from HMEC-1 cells grown to confluence on fibronectin coated coverslips and treated with either 1 nM inhibin A or 1 nM VEGFA for 30 min. (ii) Quantitation of actin stress fibers from (i) using ImageJ FibrilTool plugin. ***p<0.001; ****p<0.0001, unpaired i-test. Scale bar: 25 μm. FIG. 5B shows a (i) Western blot analysis of pMLC-2 from HMEC-1 cells upon 1 nM inhibin A treatment for indicated times. (ii) Quantitation of pMLC-2 changes in (i). FIG. 5C shows (i) schematic of VE-cadherin internalization (ii) Representative immunofluorescent images of (upper panel) cell surface labeled VE-cadherin at 4° C. detected by labeling with an extracellular domain anti-VE-cadherin antibody. Efficiency of stripping of extracellular labeled VE-cadherin with a mild acid in lower panel. (iii) Internalized VE-Cadherin at 37° C. detected with a FITC-secondary antibody in either untreated or cells treated with 1 nM inhibin A or 1 nM VEGFA after acid wash. Green arrows represent internalized VE-cadherin. Red, actin. Blue, DAPI. Quantitation of internalized VE-Cadherin at (iv) T₀ or (v) T₃₀ by Blobfinder ImageJ Plugin (Methods). *p<0.05, unpaired t-test. Scale bar: 25 μm.

Inhibin's Effects on Vascular Permeability are Mediated by ALK1 and CD105/Endoglin that Form a Stable Complex at the Cell Surface in Response to Inhibin

Previously, inhibin's effects on angiogenesis and endothelial cell signaling were demonstrated to be dependent on the TGFβ receptors ALK1 and endoglin (19). To evaluate if ALK1 and endoglin are required for inhibin's influence on vascular permeability, HMEC-1 cells were treated with Tracon 105 (TRC105), a humanized endoglin monoclonal antibody (45), or with ALK1-Fc, a human chimeric ALK1 protein (46) (FIG. 6A). At 4 h, treatment with (i) TRC105 and (ii) ALK1-Fc decreased inhibin A induced permeability by 2.2 and 1.5 times, respectively (FIG. 6A) indicating both ALK1 and endoglin are required for inhibin's effects on endothelial cell permeability.

FIG. 6A shows quantitation of endothelial cell permeability by measuring FITC-dextran changes across a HMEC-1 monolayer treated with 1 nM inhibin A in the presence or absence of (i) 100 μg/mL TRC105 or (ii) 10 ng/mL ALK1-Fc. FITC-dextran diffusion across the HMEC-1 monolayer at 4 h is presented. Mean±SD, n=4 for i and n=3 for ii. n.s. not significant; ***p<0.001; ****p<0.0001. FIG. 6B shows internalization of VE-cadherin measured by cell surface biotinylation. (i) Biotin labeling of cell surface proteins was performed on MEEC WT or MEEC ENG−/−. Internalization was induced by treatment with 1 nm Inhibin A for 30 min at 37° C. followed by stripping of cell surface biotin. Internalized VE-cadherin was detected by IP with neutravidin resin and (ii) immunoblotting with anti-VE-cadherin and (iii) quantitated as internalized VE-cadherin over input VE-cadherin normalize Wed to 37° C. control. Mean±SD, n=2. n.s. not significant; *p<0.05, unpaired t-test. FIGS. 6C and D show Patch/FRAP studies on the effect of inhibin A on endoglin-ALK1 (c) and endoglin-ALK4 (d) complex formation. COS7 cells were transfected with myc-ALK1 and HA-endoglin (c) or with (each vector HA-ALK4 and myc-endoglin) (D) (each vector alone, or together). FIG. 6C shows that after 24 h, singly transfected cells were labeled for FRAP by anti-tag Fab′ followed by fluorescent secondary Fab′ (Methods) and subjected to FRAP studies. For patch/FRAP, cells were subjected to protocol 1 of IgG-mediated patching/cross-linking (CL) (Methods), resulting in HA-endoglin patched and labeled by Alexa 488-GαR IgG (designated “CL: IgG αHA”), whereas myc-ALK1 is labeled by monovalent Fab′ (with secondary Alexa 546-GαM Fab′). In control experiments without HA-endoglin CL, the IgG labeling of the HA tag was replaced by exclusive Fab′ labeling. Where indicated, inhibin A (4 nM) was added during the fluorescent labeling step and maintained throughout the measurement. Representative FRAP curves are depicted in panels (i-iii), showing the lateral diffusion of singly expressed myc-ALK1 (i), singly expressed HA-endoglin immobilized by IgG CL (ii) and of myc-ALK1 in the presence of co-expressed and IgG-crosslinked HA-endoglin in the presence of inhibin A (iii). Panels (iv-v) depict average R_{f} (iv) and D values (v) of multiple experiments. Bars represent mean±SEM values, with the number of measurements (each conducted on a different cell) shown in each bar. Some of these numbers are lower in the D values panels, since only R_{f} can be extracted from FRAP curves yielding less than 20% recovery. Asterisks indicate significant differences between the R_{f} values of the pairs indicated by brackets (****P<1×10⁻¹⁵; ***p=1×10⁻⁹; one-way ANOVA followed by Bonferroni post-hoc test). FIG. 6D shows cells were labeled for patch/FRAP using protocol 2 (Methods), leading to immobilization (CL) of the myc-endoglin and Fab′ labeling of HA-ALK4, whose lateral diffusion was then measured by FRAP. (i) Average R_{f} values. (ii) Average) values. Bars are mean±SEM with number of measurements (n) depicted in each bar. Asterisks indicate significant differences between the R_{f} values of the pairs indicated by brackets (****p<1×10⁻¹⁵; **p=5.6×10⁻³; one-way ANOVA followed by Bonferroni post-hoc test). No significant differences were found between D values following myc-endoglin immobilization.

Whether internalization of VE-cadherin by inhibin was dependent on endoglin using mouse embryonic endothelial cells (MEEC) that are either wild type (WT) or null for endoglin expression was then evaluated (47). Cell surface biotinylation of VE-cadherin was used to quantitatively assess VE-cadherin internalization. Towards this, cell surface proteins were labeled with Sulfo-NH-SS biotin and allowed to internalize for 30 min at 37° C. in the presence or absence of inhibin followed by stripping of cell surface biotin, immunoprecipitation with neutravidin resin and immunoblotting to detect internalized biotin labeled VE-cadherin (FIG. 6B(i)). Treatment with inhibin A increased internalized VE-cadherin 1.9 times in MEEC WT compared to control (FIG. 6B(ii), similar to extents seen by immunofluorescence in HMEC-1 cells (FIG. 5C). However, in the absence of endoglin in MEEC ENG−/− cells, inhibin A did not change the internalized VE-cadherin pool (FIG. 6B(ii)). This data indicates that endoglin is essential for inhibin's effects on VE-cadherin.

Based on the significant dependency of inhibin's effects on endothelial cell permeability and VE-cadherin internalization on endoglin and ALK1 respectively (FIG. 6A, 6B), biophysically, in a sensitive and quantitative manner, the extent of the endoglin-ALK1 interaction in response to inhibin was evaluated using a patch/FRAP (fluorescence recovery after photobleaching) methodology to measure interactions between endoglin and ALK1 at the surface of live cells. This method differentiates between stable and transient interactions as described in detail previously (48). Herein, one receptor carrying an extracellular epitope tag is patched and immobilized through cross-linking with a double layer of IgGs. The effects of this immobilization on the lateral diffusion of a co-expressed, differently tagged receptor labeled exclusively with Fab′ fragments are then measured by FRAP (Methods). Stable complex formation between the two co-expressed receptors (complex lifetimes longer than the characteristic FRAP fluorescence recovery time) reduces the mobile fraction (R_{f}) of the Fab′-labeled receptor, since bleached Fab′-labeled receptors associated with immobilized receptors do not appreciably dissociate from the immobile patches during the FRAP measurement. On the other hand, transient complexes (short complex lifetimes) would reduce the apparent lateral diffusion coefficient (D), since each Fab′-labeled receptor molecule can undergo multiple association-dissociation cycles during the FRAP measurement (48). For these studies, COS7 cells were transfected with myc-ALK1, HA-endoglin or co-transfected with both, and subjected to patch/FRAP experiments in the absence or presence of 4 nM of inhibin A (FIG. 6C). FIGS. 6C(i)-(iii) depict representative FRAP curves showing the lateral diffusion of myc-ALK1 (FIG. 6C(i)), IgG-crosslinked and immobilized HA-endoglin (FIG. 6C(ii)), and myc-ALK1 co-transfected with HA-endoglin followed by IgG cross-linking of HA-endoglin in the presence of inhibin (FIG. 6C(iii)). Average values derived from multiple independent experiments are shown in (R_{f} in FIG. 6C(iv), D values in FIG. 6C(v). Singly expressed myc-ALK1 had lateral mobility resembling other TGF-β superfamily receptors (49), which was insensitive to inhibin treatment (FIGS. 6C(i) and (iv)). Immobilization of HA-endoglin (FIGS. 6C(ii) and (iv) reduced R_{f} of myc-ALK1 by about 45%, and the presence of inhibin increased this reduction significantly (from 45% to 70% reduction) (FIGS. 6C(iii)-(iv)). Under all these conditions, the lateral diffusion coefficient (D) of myc-ALK1 was not significantly affected (FIG. 6C(v)), indicating that endoglin and ALK1 form stable complexes at the plasma membrane which are enhanced and stabilized by inhibin.

Previous studies indicate that inhibinα may bind to ALK4 (50), an established Type I receptor for the activin family of proteins (8). Oatch/FRAP was employed to determine the interactions between endoglin and ALK4 and to examine whether inhibin A enhanced these interactions. To this end, HA-ALK4, myc-endoglin or both were expressed in COS7 cells, and subjected them to patch/FRAP studies on the lateral diffusion of HA-ALK4 without and with IgG cross-linking of myc-endoglin, and with or without inhibin A. In the absence of inhibin A, endoglin and ALK4 exhibited significant stable interactions, as demonstrated by the reduction in R_{f} of HA-ALK4 upon immobilization of myc-endoglin (40% reduction in R_{f}, with no effect on the D value) (FIGS. 6D(i)-(ii)). However, in contrast to the observations with endoglin-ALK1 complexes, the interactions between endoglin and ALK4 were weakened in the presence of inhibin A (the reduction in R_{f} decreased to 20%) (FIG. 6D(i)). Taken together, these results indicate that inhibin shifts the balance of endoglin complexes from interactions with ALK4 to interactions with ALK1, both of which (endoglin and ALK1) are required for inhibin-mediated vascular permeability.

### Inhibin Promotes Hypoxia Induced Tumor Growth In Vivo Through Alterations in Permeability and Angiogenesis

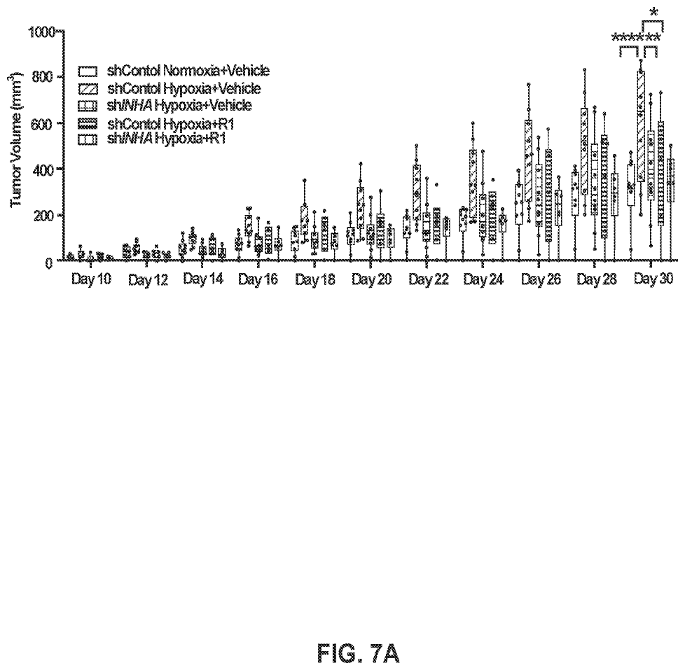

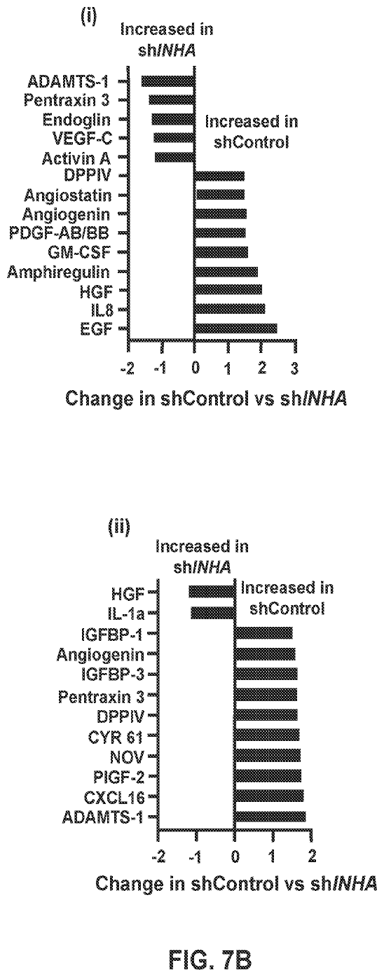

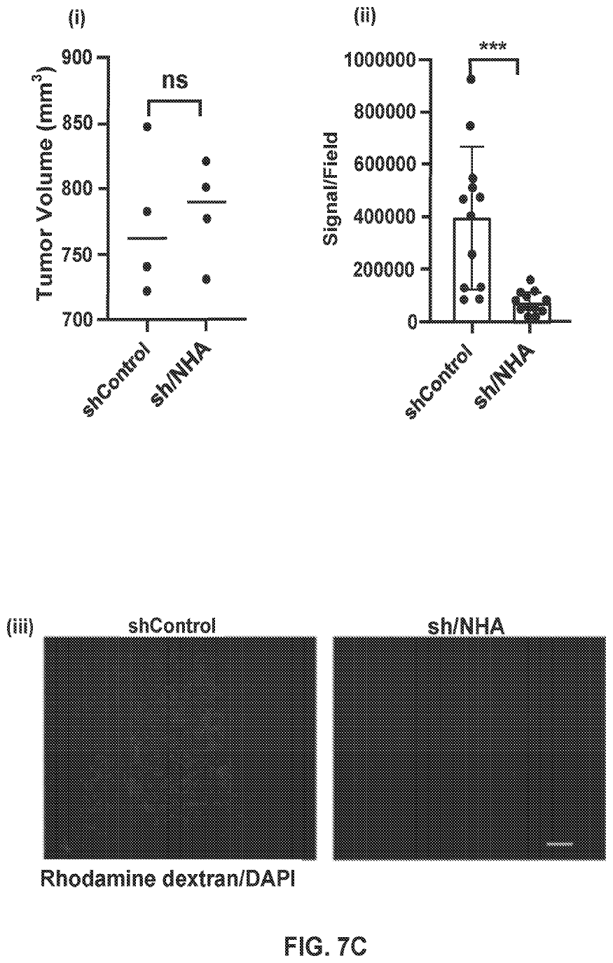

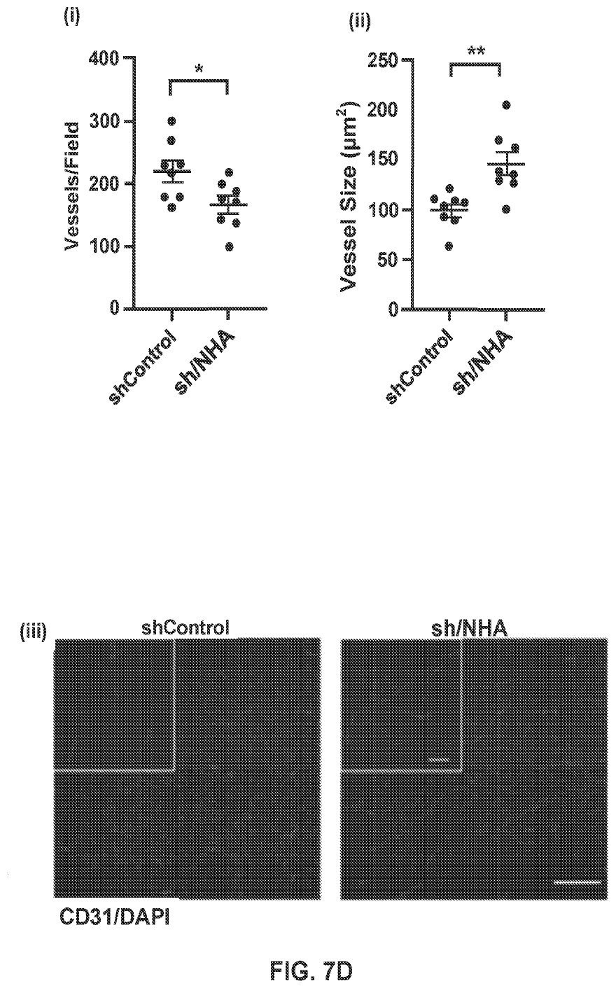

The significance of hypoxia in ovarian cancer is well documented and increased ascites accumulation occurs in tumor bearing mice in the presence of inhibin (19). To precisely define the contribution of inhibin to hypoxia induced tumor growth and angiogenesis, the effects of pre-exposure to hypoxia on tumor growth was evaluated in a subcutaneous model in vivo, a model that allows for quantitative analysis of the vasculature in tumors (51). HEY pLKO.1 control vector (shControl) cells were pre-exposed to hypoxia (0.2% O₂) for 24 h or kept under normoxia followed by injection into the right flank of Ncr nude mice. Tumors were measured throughout and harvested after 30 days (n=10 mice). HEY cells pre-exposed to hypoxia produced rapid growing tumors compared to those that originated from normoxia grown cells (FIG. 7A, purple versus gray). In parallel, two methods were used to perturb inhibin: (1) shRNA knockdown of INHA in HEY cells and (2) intraperitoneal administration of anti-inhibinα antibody (R1). R1 is a human antibody (41) and consistent with this, no overall toxicity was noted in pilot toxicity studies that utilized daily injections of R1. shINHA cells exposed to hypoxia maintained their knockdown to INHA at the end of the study and produced tumors with significantly slower growth rates than shControl hypoxia tumors (FIG. 7A, blue versus gray). In complementary findings, hypoxia exposed tumor cells had significantly reduced tumor growth upon receiving treatment with the R1 antibody when compared to tumors in mice that received vehicle only (FIG. 7A, red versus gray, n=6 for R1 treated mice). The group receiving anti-inhibinα (R1) grew at a similar rate as the shINHA hypoxia tumors (FIG. 7A, red versus blue). In mice with shINHA tumors, treatment with R1 further reduced tumor growth albeit moderately compared to vehicle shINHA (FIG. 7A, blue versus green). These data indicate that perturbation of inhibin through shRNA targeting and anti-inhibin antibody treatment reduces tumor growth. The effect of shINHA on the angiogenic cytokine profile of the tumors using a proteome array of 55 different human angiogenesis targets was then determined. The most upregulated proteins in control tumors compared to shINHA tumors were a subset of pro-angiogenic cytokines: IL-8 (2.5 times) and EGF (2.1 times) (FIG. 7B(i)) indicating a pro-angiogenic profile of the tumor cells in the presence of inhibin. In contrast, the shINHA hypoxia tumors showed increases in proteins including ADAMTS-1 (1.6 times) and Pentraxin-3 (1.3 times), indicating an anti-angiogenic profile in shINHA tumor cells as both have been demonstrated to be anti-angiogenic (52-53). Activin A and endoglin were also found to be elevated in shINHA tumors (FIG. 7B(i)). To complement the human tumor array, changes in the mouse angiogenic proteome were analyzed as well to delineate any host differences in response to shControl and shINHA tumor cells. Host cells also upregulated significantly more pro-angiogenic proteins, including CXCL 16 (54), PIGF-2 (55), and NOV (56) in shControl tumors compared to shINHA tumors (FIG. 7B(ii)). Taken together, these data suggest that altering inhibin in the tumors results in a change in the balance of angiogenic factors leading to a significant reduction in pro-angiogenic factors and slower overall tumor growth.

FIG. 7A shows growth curves of subcutaneously implanted HEY shControl or shINHA tumors exposed to either normoxia or hypoxia (0.2% O₂) 24 h prior to injection. 10 mg/kg R1 antibody or vehicle control was intraperitoneally injected three times a week. Data shown as box plots where center line is median, box limits are upper and lower quartile, n=10 for vehicle and n=6 for R1 receiving groups. **p<0.01; ****p<0.0001, Two-way ANOVA followed by Tukey's multiple comparison test. FIG. 7B shows fold change of proteins most altered in shControl and shINHA tumors (a) using the (i) human or (ii) mouse angiogenesis proteome array. (n=2 tumors per group). FIG. 7C shows (i) Average tumor volume of HEY shControl or shINHA subcutaneous tumors used for analysis of vasculature and permeability in ii and iii. Mean±SEM, n=4. (ii) Quantitation of extravasated rhodamine-dextran (red) shown as signal per 10× field from tumors in FIG. 7(c)(i) (Methods). Mean±SD. n=12 fields from 4 tumors. ***p<0.001, unpaired f-test. (iii) Representative images of rhodamine-dextran (red) extravasation into either shControl or shINHA subcutaneous tumors from c.i Scale bar: 100 μm. FIG. 7D shows (i-ii) quantitation of average (i) vessel number and (ii) size in a 10× field using ImageJ (Methods). Mean±SD. in =8 which represents averages of 8 fields in four tumors from c.i. (iii) Representative images of CD-31 (red) staining in HEY shControl and shINHA subcutaneous tumors from FIG. 7C(i). Scale bar: 100 μm, insets scale bar: 20 μm. *p<0.05; **p<0.01, unpaired t-test.

To rule out whether the reduction in tumor growth in shINHA cells was due to slower proliferation of tumor cells, growth rate of HEY shINHA and HEY shControl was evaluated in culture under hypoxia for 3 days. No significant change was observed, suggesting that the major effect of inhibin on tumor growth are likely through effects on the tumor vasculature due to the effects of hypoxia regulated inhibin on angiogenesis and vascular permeability in vitro (FIGS. 4-5). The effect of inhibin on the tumor vasculature and associated permeability changes was thus determined as a contributing factor to the altered tumor growth in shINHA and antibody treated hypoxia tumors (FIG. 7A). To this end, HEY shControl or shINHA cells pre-exposed to hypoxia for 24 h were injected subcutaneously into the right flank of Ncr nude mice (n=4 mice) and tumors in all groups were harvested upon reaching 700-850 mm³ (FIG. 7C(i)) to eliminate any tumor size effects on angiogenesis. These tumors (FIG. 7C(i)) were evaluated for changes in vascular permeability by visualization of a rhodamine-dextran dye that leaks from the blood vessels into the tumors when administered into mice prior to sacrifice. Rhodamine-dextran was present at 5.5 times higher levels in shControl tumors compared to shINHA tumors, indicating higher vascular permeability within the tumors in the presence of inhibin (FIGS. 7C(ii)-(iii). To further characterize the differences in the vasculature between shControl and shINHA tumors, blood vessels were stained with CD-31 to evaluate vessel number and size (FIG. 7D). An increase in the total number of blood vessels in shControl tumors occurred compared to shINHA tumors (FIGS. 7D(i) and (iii)). Quantitation of the size of the vessels revealed significantly smaller vessels in shControl tumors as compared to the shINHA tumors (FIGS. 7D(ii) and (iii)). These data together demonstrate that reducing inhibin in the tumor decreases vascular leakiness, alters vessel size and numbers and promotes more normalized vasculature in the tumors.

Publications cited herein and the material for which they are cited are hereby specifically incorporated by reference in their entireties.

In the following, further embodiments are described to facilitate the understanding of the invention:
Embodiment 1: A method of reducing metastasis and/or tumor growth in a subject with cancer, the method comprising administering an effective amount of an inhibin antibody to the subject with cancer.
Embodiment 2: The method according to Embodiment 1 or 2, wherein the cancer is selected from the group consisting of breast cancer, ovarian cancer, melanoma, squamous cell carcinoma, bladder cancer, lung cancer, testicular cancer, kidney cancer, colorectal cancer, and head and neck cancer.
Embodiment 3: The method according to Embodiment 1 or 2, wherein the cancer is ovarian cancer.
Embodiment 4: The method according to any one of Embodiments 1-3, wherein the method further comprises concurrent administration of an anti-angiogenic.
Embodiment 5: The method according to any one of Embodiments 1-4, wherein the method further comprises administration of an anti-angiogenic prior to administration of the inhibin antibody.
Embodiment 6: The method according to any one of Embodiments 1-4, wherein the method further comprises administration of an anti-angiogenic after administration of the inhibin antibody.
Embodiment 7: The method according to any one of Embodiments 1-6, wherein the anti-inhibin is administered to a subject with tumors resistant to treatment with TRC105 and Becacizumab.
Embodiment 8: The method according to any one of Embodiments 1-7, wherein the tumor growth is hypoxia induced tumor growth.
Embodiment 9: The method according to any one of Embodiments 1-8, wherein the inhibin antibody is anti-inhibin R1 antibody.
Embodiment 10: The method according to any one of Embodiments 1-9, wherein the inhibin antibody is PO23 anti-inhibin antibody.
Embodiment 11: The method according to any one of Embodiments 1-10, wherein the subject is pre-menopausal.
Embodiment 12: The method according to any one of Embodiments 1-10, wherein the subject is post-menopausal.
Embodiment 13: The method according to any one of Embodiments 1-12, wherein the method is for reducing metastasis.
Embodiment 14: The method according to any one of Embodiments 1-12, wherein the method is for reducing tumor growth.
Embodiment 15: The method according to any one of Embodiments 1-12, wherein the method is for reducing metastasis in a subject with gynecological cancer, such as ovarian cancer, or breast cancer.
Embodiment 16: The method according to any one of Embodiments 1-12, wherein the method is for reducing tumor growth in a subject with gynecological cancer, such as ovarian cancer, or breast cancer.
Embodiment 17: The method according to any one of Embodiments 1-16, wherein the method further comprises treating the subject with chemotherapy.
Embodiment 18: The method according to any one of Embodiments 1-17, wherein the subject is a mammal.
Embodiment 19: The method according to any one of Embodiments 1-18, wherein the subject is a human.
Embodiment 20: The method according to any one of Embodiments 1-19, wherein the effective amount is from 0.01 to 200 mg/kg.
Embodiment 21: The method according to any one of Embodiments 1-20, wherein the method further comprises concurrent administration of an HIF targeted therapy.

### LIST OF REFERENCES

1. Viallard, C. & Larrivee, B. Tumor angiogenesis and vascular normalization: alternative therapeutic targets. Angiogenesis 20, 409-426 (2017).
2. Adam, R. A. & Adam, Y. G. Malignant ascites: past, present, and future. J. Am. Coll. Surg. 198, 999-1011 (2004).
3. Monk, B. J., Minion, L. E. & Coleman, R. L. Anti-angiogenic agents in ovarian cancer: past, present, and future. Ann. Oncol. 27, i33-i39 (2016).
4. Impact of selective anti-BMP9 treatment on tumor cells and tumor angiogenesis. Mol. Oncol. (2016).
5. Goumans, M. J., Liu, Z. & Ten Dijke, P. TGF-beta signaling in vascular biology and dysfunction. Cell Res. 19, 116-127 (2009).
6. Maeshima, K., Maeshima, A., Hayashi, Y., Kishi, S. & Kojima, I. Crucial role of activin a in tubulogenesis of endothelial cells induced by vascular endothelial growth factor. Endocrinology 145, 3739-3745 (2004).
7. Panopoulou, E. et al. Activin A suppresses neuroblastoma xenograft tumor growth via antimitotic and antiangiogenic mechanisms. Cancer Res. 65, 1877-1886 (2005).
8. Makanji, Y. et al. Inhibin at 90: from discovery to clinical application, a historical review. Endocr. Rev. 35, 747-794 (2014).
9. Meunier, H., Rivier, C., Evans, R. M. & Vale, W. Gonadal and extragonadal expression of inhibin alpha, beta A, and beta B subunits in various tissues predicts diverse functions. Proc. Natl Acad. Sci. USA 85, 247-251 (1988).
10. Woodruff, T. K. & Mayo, K. E. Regulation of inhibin synthesis in the rat ovary. Annu Rev. Physiol. 52, 807-821 (1990).
11. Tsonis, C. G., Hillier, S. G. & Baird, D. T. Production of inhibin bioactivity by human granulosa-lutein cells: stimulation by LH and testosterone in vitro. J. Endocrinol. 112, R11-R14 (1987).
12. Walton, K. L., Makanji, Y., Robertson, D. M. & Harrison, C. A. The synthesis and secretion of inhibins. Vitam. Horm. 85, 149-184 (2011).
13. Overlie, I. et al. Inhibin A and B as markers of menopause: a five-year prospective longitudinal study of hormonal changes during the menopausal transition. Acta Obstet. Gynecol. Scand. 84, 281-285 (2005).
14. Shanbhag, S. A., Sheth, A. R., Nanivadekar, S. A. & Sheth, N. A. Immunoreactive inhibin-like material in serum and gastric juice of patients with benign and malignant diseases of the stomach. Br. J. Cancer 51, 877-882 (1985).
15. Balanathan, P. et al. Elevated level of inhibin-alpha subunit is pro-tumourigenic and pro-metastatic and associated with extracapsular spread in advanced prostate cancer. Br. J. Cancer 100, 1784-1793 (2009).
16. McCluggage, W. G., Maxwell, P., Patterson, A. & Sloan, J. M. Immunohistochemical staining of hepatocellular carcinoma with monoclonal antibody against inhibin. Histopathology 30, 518-522 (1997).
17. Walentowicz, P. et al. Serum inhibin A and inhibin B levels in epithelial ovarian cancer patients. PLoS One 9, e90575 (2014).
18. Robertson, D. M., Pruysers, E. & Jobling, T. Inhibin as a diagnostic marker for ovarian cancer. Cancer Lett. 249, 14-17 (2007).
19. Singh, P. et al. Inhibin is a novel paracrine factor for tumor angiogenesis and metastasis. Cancer Res. 78, 2978-2989 (2018).
20. Listik, E. et al. A bioinformatic analysis of the inhibin-betaglycan-endoglin/CD105 network reveals prognostic value in multiple solid tumors. PLoS One 16, e0249558 (2021).
21. Rankin, E. B. & Giaccia, A. J. Hypoxic control of metastasis. Science 352, 175-180 (2016).
22. Ahmed, N. & Stenvers, K. L. Getting to know ovarian cancer ascites: opportunities for targeted therapy-based translational research. Front Oncol. 3, 256 (2013).
23. Arola, J. et al. Expression of inhibin alpha in adrenocortical tumours reflects the hormonal status of the neoplasm. J. Endocrinol. 165, 223-229 (2000).
24. Ziyad, S. & Iruela-Arispe, M. L. Molecular mechanisms of tumor angiogenesis. Genes Cancer 2, 1085-1096 (2011).
25. Bu, S. et al. Epithelial ovarian cancer stem-like cells are resistant to the cellular lysis of cytokine-induced killer cells via HIF1A-mediated downregulation of ICAM-1. Int J. Oncol. 55, 179-190 (2019).
26. Kawata, M., Sekiya, S., Kera, K., Kimura, H. & Takamizawa, H. Neural rosette formation within in vitro spheroids of a clonal human teratocarcinoma cell line, PA-1/NR: role of extracellular matrix components in the morphogenesis. Cancer Res. 51, 2655-2669 (1991).
27. Masiello, T. et al. A dynamic culture method to produce ovarian cancer spheroids under physiologically-relevant shear stress. Cells (2018).
28. Groome, N. P. et al. Detection of dimeric inhibin throughout the human menstrual cycle by two-site enzyme immunoassay. Clin. Endocrinol. (Oxf) 40, 717-723 (1994).
29. NCI. Cancer Stat Facts: Ovarian Cancer (2019).
30. Kim, K. S. et al. Hypoxia enhances lysophosphatidic acid responsiveness in ovarian cancer cells and lysophosphatidic acid induces ovarian tumor metastasis in vivo. Cancer Res. 66, 7983-7990 (2006).
31. Carreau, A., El Hafny-Rahbi, B., Matejuk, A., Grillon, C. & Kieda, C. Why is the partial oxygen pressure of human tissues a crucial parameter? Small molecules and hypoxia. J. Cell Mol. Med. 15, 1239-1253 (2011).
32. Cerami, E. et al. The cBio cancer genomics portal: an open platform for exploring multidimensional cancer genomics data. Cancer Disco. 2, 401-404 (2012).
33. Gao, J. et al. Integrative analysis of complex cancer genomics and clinical profiles using the cBioPortal. Sci. Signal 6, pl1 (2013).
34. Buffa, F. M., Harris, A. L., West, C. M. & Miller, C. J. Large meta-analysis of multiple cancers reveals a common, compact and highly prognostic hypoxia metagene. Br. J. Cancer 102, 428-435 (2010).
35. Winter, S. C. et al. Relation of a hypoxia metagene derived from head and neck cancer to prognosis of multiple cancers. Cancer Res. 67, 3441-3449 (2007).
36. Yuan, Y., Hilliard, G., Ferguson, T. & Millhorn, D. E. Cobalt inhibits the interaction between hypoxia-inducible factor-alpha and von Hippel-Lindau protein by direct binding to hypoxia-inducible factor-alpha. J. Biol. Chem. 278, 15911-15916 (2003).
37. Dengler, V. L., Galbraith, M. & Espinosa, J. M. Transcriptional regulation by hypoxia inducible factors. Crit. Rev. Biochem. Mol. Biol. 49, 1-15 (2014).
38. Yan, Q., Bartz, S., Mao, M., Li, L. & Kaelin, W. G. Jr. The hypoxia-inducible factor 2alpha N-terminal and C-terminal transactivation domains cooperate to promote renal tumorigenesis in vivo. Mol. Cell Biol. 27, 2092-2102 (2007).
39. Simko, V. et al. Hypoxia induces cancer-associated cAMP/PKA signalling through HIF-mediated transcriptional control of adenylyl cyclases VI and VII. Sci. Rep. 7, 10121 (2017).
40. Depoix, C. L., Debieve, F. & Hubinont, C. Inhibin alpha gene expression in human trophoblasts is regulated by interactions between TFAP2 and cAMP signaling pathways. Mol. Reprod. Dev. 81, 1009-1018 (2014).
41. Robertson, D. M. et al. Development of an inhibin alpha subunit ELISA with broad specificity. Mol. Cell Endocrinol. 180, 79-86 (2001).
42. Baratchi, S. et al. Molecular sensors of blood flow in endothelial cells. Trends Mol. Med. 23, 850-868 (2017).
43. Shenoy, A. K. & Lu, J. Cancer cells remodel themselves and vasculature to overcome the endothelial barrier. Cancer Lett. 380, 534-544 (2016).
44. Dejana, E. Endothelial cell-cell junctions: happy together. Nat. Rev. Mol. Cell Biol. 5, 261-270 (2004).
45. Rosen, L. S. et al. A phase I first-in-human study of TRC105 (Anti-endoglin antibody) in patients with advanced cancer. Clin. Cancer Res. 18, 4820-4829 (2012).
46. Mitchell, D. et al. ALK1-Fc inhibits multiple mediators of angiogenesis and suppresses tumor growth. Mol. Cancer Ther. 9, 379-388 (2010).
47. Pece-Barbara, N. et al. Endoglin null endothelial cells proliferate faster and are more responsive to transforming growth factor beta1 with higher affinity receptors and an activated AlkI pathway. J. Biol. Chem. 280, 27800-27808 (2005).
48. Pomeraniec, L., Hector-Greene, M., Ehrlich, M., Blobe, G. C. & Henis, Y. I. Regulation of TGF-beta receptor hetero-oligomerization and signaling by endoglin. Mol. Biol. Cell 26, 3117-3127 (2015).
49. Rechtman, M. M., Nakaryakov, A., Shapira, K. E., Ehrlich, M. & Henis, Y. I. Different domains regulate homomeric and heteromeric complex formation among type I and type II transforming growth factor-beta receptors. J. Biol. Chem. 284, 7843-7852 (2009).
50. Zhu, J. et al. Inhibin α-subunit N terminus interacts with activin type IB receptor to disrupt activin signaling. J. Biol. Chem. 287, 8060-8070 (2012).
51. Forster, J. C., Harriss-Phillips, W. M., Douglass, M. J. & Bezak, E. A review of the development of tumor vasculature and its effects on the tumor microenvironment. Hypoxia (Auckl.) 5, 21-32 (2017).
52. Lee, N. V. et al. ADAMTS1 mediates the release of antiangiogenic polypeptides from TSP1 and 2. EMBOJ. 25, 5270-5283 (2006).
53. Presta, M., Foglio, E., Churruca Schuind, A. & Ronca, R. Long Pentraxin-3 modulates the angiogenic activity of fibroblast growth factor-2. Front Immunol. 9, 2327 (2018).
54. Korbecki, J. et al. The role of CXCL16 in the pathogenesis of cancer and other diseases. Int J Mol Sci. (2021).
55. De Falco, S. The discovery of placenta growth factor and its biological activity. Exp. Mol. Med. 44, 1-9 (2012).
56. Lin, C. G. et al. CCN3 (NOV) is a novel angiogenic regulator of the CCN protein family. J. Biol. Chem. 278, 24200-24208 (2003).
57. Merfeld-Clauss, S., Lu, H., Wu, X., March, K. L. & Traktuev, D. O. Hypoxia-induced activin A diminishes endothelial cell vasculogenic activity. J. Cell Mol. Med. 22, 173-184 (2018).
58. Alam, H. et al. Follicle-stimulating hormone activation of hypoxia-inducible factor-1 by the phosphatidylinositol 3-kinase/AKT/Ras homolog enriched in brain (Rheb)/mammalian target of rapamycin (mTOR) pathway is necessary for induction of select protein markers of follicular differentiation. J. Biol. Chem. 279, 19431-19440 (2004).
59. Depoix, C. L., de Selliers, I., Hubinont, C. & Debieve, F. HIF1A and EPAS1 potentiate hypoxia-induced upregulation of inhibin alpha chain expression in human term cytotrophoblasts in vitro. Mol. Hum. Reprod. 23, 199-209 (2017).
60. Zhang, H., Kong, Q., Wang, J., Jiang, Y. & Hua, H. Complex roles of cAMP-PKA-CREB signaling in cancer. Exp. Hematol. Oncol. 9, 32 (2020).
61. Balanathan, P. et al. Epigenetic regulation of inhibin alpha-subunit gene in prostate cancer cell lines. J. Mol. Endocrinol. 32, 55-67 (2004).
62. Hofland, J. et al. Inhibin alpha-subunit (INHA) expression in adrenocortical cancer is linked to genetic and epigenetic INHA promoter variation. PLoS One 9, e104944 (2014).
63. D'Anna, F. et al. DNA methylation repels binding of hypoxia-inducible transcription factors to maintain tumor immunotolerance. Genome Biol. 21, 182 (2020).
64. Benn, A., Bredow, C., Casanova, I., Vukiičević, S. & Knaus, P. VE-cadherin facilitates BMP-induced endothelial cell permeability and signaling. J. Cell Sci. 129, 206-218 (2016).
65. Aykul, S. et al. Activin A forms a non-signaling complex with ACVR1 and type II Activin/BMP receptors via its finger 2 tip loop. Elife (2020).
66. Olsen, O. E. et al. Activin A inhibits BMP-signaling by binding ACVR2A and ACVR2B. Cell Commun. Signal 13, 27 (2015).
67. Bai, S. et al. CD105 is expressed in ovarian cancer precursor lesions and is required for metastasis to the ovary. Cancers (Basel) (2019).
68. Qin, J. et al. Hypoxia-inducible factor 1 alpha promotes cancer stem cells-like properties in human ovarian cancer cells by upregulating SIRT1 expression. Sci. Rep. 7, 10592 (2017).
69. Milane, L., Duan, Z. & Amiji, M. Role of hypoxia and glycolysis in the development of multi-drug resistance in human tumor cells and the establishment of an orthotopic multi-drug resistant tumor model in nude mice using hypoxic pre-conditioning. Cancer Cell Int. 11, 3 (2011).
70. Jain, R. K. Antiangiogenesis strategies revisited: from starving tumors to alleviating hypoxia. Cancer Cell 26, 605-622 (2014).
71. Liu, Y. et al. Modulation of circulating protein biomarkers in cancer patients receiving Bevacizumab and the anti-endoglin antibody, TRC105. Mol. Cancer Ther. 17, 2248-2256 (2018).
72. Huang, Z. et al. Targeting dormant ovarian cancer cells in vitro and in an in vivo mouse model of platinum resistance. Mol. Cancer Ther. 20, 85-95 (2021).
73. Nakayama, J., Raines, T. A., Lynch, K. R. & Slack-Davis, J. K. Decreased peritoneal ovarian cancer growth in mice lacking expression of lipid phosphate phosphohydrolase 1. PLoS One 10, e0120071 (2015).
74. Martins-Green, M., Petreaca, M. & Yao, M. An assay system for in vitro detection of permeability in human “endothelium”. Methods Enzymol. 443, 137-153 (2008).
75. Boudaoud, A. et al. FibrilTool, an ImageJ plug-in to quantify fibrillar structures in raw microscopy images. Nat. Protoc. 9, 457-463 (2014).
76. Allalou, A. & Wahlby, C. BlobFinder, a tool for fluorescence microscopy image cytometry. Comput Methods Prog. Biomed. 94, 58-65 (2009).
77. Lee, N. Y., Kirkbride, K. C., Sheu, R. D. & Blobe, G. C. The transforming growth factor-beta type III receptor mediates distinct subcellular trafficking and downstream signaling of activin-like kinase (ALK)3 and ALK6 receptors. Mol. Biol. Cell 20, 4362-4370 (2009).
78. Henis, Y. I., Moustakas, A., Lin, H. Y. & Lodish, H. F. The types II and III transforming growth factor-beta receptors form homo-oligomers. J. Cell Biol. 126, 139-154 (1994).
79. Gilboa, L., Wells, R. G., Lodish, H. F. & Henis, Y. I. Oligomeric structure of type I and type II transforming growth factor beta receptors: homodimers form in the ER and persist at the plasma membrane. J. Cell Biol. 140, 767-777 (1998).
80. Horst et al. Hypoxia-induced inhibin promotes tumor growth and vascular permeability in ovarian cancers. Commun Biol. 2022 Jun. 2; 5(1):536.

1. A method of reducing metastasis and/or tumor growth and/or changes in the vasculature in a subject with cancer, the method comprising administering an effective amount of an inhibin antibody to the subject with cancer.

2. The method according to claim 1, wherein the cancer is selected from the group consisting of breast cancer, gynecological cancer, prostate, melanoma, squamous cell carcinoma, bladder cancer, lung cancer, testicular cancer, kidney cancer, colorectal cancer, and head and neck cancer.

3. The method according to claim 1, wherein the cancer is gynecological.

4. The method according to claim 1, wherein the method further comprises concurrent administration of an anti-angiogenic and/or a chemotherapeutic.

5. The method according to claim 1, wherein the method further comprises administration of an anti-angiogenic or chemotherapeutic prior to administration of the inhibin antibody.

6. The method according to claim 1, wherein the method further comprises administration of an anti-angiogenic and/or chemotherapeutic after administration of the inhibin antibody.

7. The method according to claim 1, wherein the anti-inhibin is administered to a subject with tumors resistant to treatment with TRC105 and Becacizumab.

8. The method according to claim 1, wherein the tumor growth is hypoxia induced tumor growth.

9. The method according to claim 1, wherein the inhibin antibody is anti-inhibin R1 antibody.

10. The method according to claim 1, wherein the inhibin antibody is PO23 anti-inhibin antibody.

11. The method according to claim 1, wherein the subject is pre-menopausal.

12. The method according to claim 1, wherein the subject is post-menopausal.

13. The method according to claim 1, wherein the method is for reducing metastasis.

14. The method according to claim 1, wherein the method is for reducing tumor growth.

13. The method according to claim 1, wherein the method is for changing the vasculature.

15. The method according to claim 1, wherein the method is for reducing metastasis in a subject with gynecological or breast cancer.

16. The method according to claim 1, wherein the method is for reducing tumor growth in a subject with gynecological or breast cancer.

17. The method according to claim 1, wherein the method further comprises treating the subject with chemotherapy.

18. The method according claim 1, wherein the subject is a mammal.

19. The method according to claim 1, wherein the subject is a human.

20. The method according to claim 1, wherein the effective amount is from 0.01 to 200 mg/kg.

21. The method according to claim 1, wherein the method further comprises concurrent administration of an HIF targeted therapy.


SEQUENCE LISTING (26 sequences)

SEQ ID NO: 1 - DNA, 17 bp, synthetic construct
  source 1..17: mol_type = other DNA
agatggcggaggtgcag

SEQ ID NO: 2 - DNA, 20 bp, synthetic construct
  source 1..20: mol_type = other DNA
ggcccagcagtacctgttta

SEQ ID NO: 3 - DNA, 20 bp, synthetic construct
  source 1..20: mol_type = other DNA
cgctcaactcccctgatgtc

SEQ ID NO: 4 - DNA, 20 bp, synthetic construct
  source 1..20: mol_type = other DNA
gggtacacgatccaccgttc

SEQ ID NO: 5 - DNA, 19 bp, synthetic construct
  source 1..19: mol_type = other DNA
cgccaaccacaacatgcag

SEQ ID NO: 6 - DNA, 19 bp, synthetic construct
  source 1..19: mol_type = other DNA
gctccacgaaggatgccac

SEQ ID NO: 7 - DNA, 20 bp, synthetic construct
  source 1..20: mol_type = other DNA
gccatccaatcgagaccctg

SEQ ID NO: 8 - DNA, 21 bp, synthetic construct
  source 1..21: mol_type = other DNA
tgattgttggactcctcagtg

SEQ ID NO: 9 - DNA, 20 bp, synthetic construct
  source 1..20: mol_type = other DNA
cgtcaggaggcacacactta

SEQ ID NO: 10 - DNA, 22 bp, synthetic construct
  source 1..22: mol_type = other DNA
cacatttgacagacagggcaat

SEQ ID NO: 11 - DNA, 22 bp, synthetic construct
  source 1..22: mol_type = other DNA
gaacgggtatgtggagatagag

SEQ ID NO: 12 - DNA, 18 bp, synthetic construct
  source 1..18: mol_type = other DNA
tgttcctgactcggcaaa

SEQ ID NO: 13 - DNA, 20 bp, synthetic construct
  source 1..20: mol_type = other DNA
gcgcgtttccgaaatcatca

SEQ ID NO: 14 - DNA, 20 bp, synthetic construct
  source 1..20: mol_type = other DNA
aggttctggttgccttcgtt

SEQ ID NO: 15 - DNA, 21 bp, synthetic construct
  source 1..21: mol_type = other DNA
tgactcctgttttgaaccagc

SEQ ID NO: 16 - DNA, 23 bp, synthetic construct
  source 1..23: mol_type = other DNA
ctgctcacgaagtttatccacat

SEQ ID NO: 17 - DNA, 20 bp, synthetic construct
  source 1..20: mol_type = other DNA
caaggttgttcggctgaagc

SEQ ID NO: 18 - DNA, 20 bp, synthetic construct
  source 1..20: mol_type = other DNA
gctgtcactgcctggtactt

SEQ ID NO: 19 - DNA, 20 bp, synthetic construct
  source 1..20: mol_type = other DNA
aggaagatgtctcccaggct

SEQ ID NO: 20 - DNA, 20 bp, synthetic construct
  source 1..20: mol_type = other DNA
gttgggatggccggaataca

SEQ ID NO: 21 - DNA, 21 bp, synthetic construct
  source 1..21: mol_type = other DNA
acgacagaaggagagcagaag

SEQ ID NO: 22 - DNA, 21 bp, synthetic construct
  source 1..21: mol_type = other DNA
atgtccaccagggtctcaatc

SEQ ID NO: 23 - DNA, 20 bp, synthetic construct
  source 1..20: mol_type = other DNA
gggatgttcaggtccatcag

SEQ ID NO: 24 - DNA, 22 bp, synthetic construct
  source 1..22: mol_type = other DNA
cacactgtagttgtgcagtcaa

SEQ ID NO: 25 - DNA, 20 bp, synthetic construct
  source 1..20: mol_type = other DNA
cctcgttcacccagaaggtc

SEQ ID NO: 26 - DNA, 20 bp, synthetic construct
  source 1..20: mol_type = other DNA
gattccggcgtctacgtgtg